(19) United States
(12) Patent Application Publication
(10) Pub. No.: US 2012/0263827 A1
(43) Pub. Date: Oct. 18, 2012

(54) BACTERIAL ISOLATE, METHODS OF ISOLATING BACTERIAL ISOLATES AND METHODS FOR DETOXIFICATION OF TRICHOTHECENE MYCOTOXINS

(75) Inventors: Ting Zhou, Guelph (CA); Jianwei He, Ottawa (CA)

(73) Assignees: and Agri-Food, Saskatoon, SK (CA); Her Majesty The Queen in Right of Canada, as represented by The Minister of Agriculture

(21) Appl. No.: 13/500,239

(22) PCT Filed: Oct. 6, 2010
(86) PCT No.: PCT/CA2010/001594

§ 371 Date: Jun. 28, 2012

(30) Foreign Application Priority Data
Oct. 6, 2009 (US) 61249023

(51) Int. Cl. C12N 1/20; A23L 3/3571; C12Q 1/02; A01N 63/00; A01P 1/00

(52) U.S. Cl. 426/61; 435/252.1; 424/93.4; 435/29; 426/532

(57) ABSTRACT

The invention provides a bacterial isolate defined by accession number 040408-1 filed with the International Depository Authority of Canada. The bacteria are capable of detoxifying trichothecene mycotoxins. Also provided are compositions comprising the bacteria and methods of preventing or treating food or foodstuffs that are contaminated or susceptible to contamination with trichothecene mycotoxins. Kits are also provided.

### FIELD OF INVENTION

The present invention relates to detoxification microorganisms. More specifically, the present invention relates to bacterial isolates and methods for detoxifying mycotoxins.

### BACKGROUND OF THE INVENTION

Approximately 25% of the world's food crops are contaminated with mycotoxins every year, creating an ongoing, serious threat to human health and food and livestock industries. Control of mycotoxins is a global challenge due to their high toxicity to animals and humans and their widespread occurrence in agricultural commodities.

Trichothecene mycotoxins represent one of the most important mycotoxin classes comprising naturally occurring metabolites produced primarily by Fusarium and other species of fungi (Stachybotrys, Myrothecium, and Trichothecium) on a variety of cereal grains. The mycotoxins are known to be associated with several diseases in animals and humans (Ueno, 1983; Pittet, 1998; D'Mello et al., 1999; Placinta et al., 1999; DeVries et al., 2002; Conková et al., 2003; Eriksen and Pettersson, 2004; Desjardins, 2006).

Deoxynivalenol (DON or vomitoxin) is a specific trichothecene mycotoxin which is frequently encountered in human foods. DON is associated primarily with Fusarium graminearum Schwabe (teleomorph Gibberella zeae (Schwein.) Petch.) (Nelson, 2002) and these fungi can produce DON under a wide range of conditions both in the field and post-harvest (Ramirez et al., 2006).

Control of mycotoxin contamination has been one of the major challenges facing the cereal industry. A survey conducted in Eastern Canada during 1991 to 1998 found that maize had the highest incidence of DON contaminated samples (0.1 mg/kg and over), which was 90%, followed by wheat, 82%, and barley 73%. In 2003, DON was detected in 63% of samples obtained from cereal-based infant foods from the Canadian retail market. Many outbreaks of acute human diseases have been attributed to consumption of Fusarium-contaminated grains and, more recently, to the presence of DON at reported concentrations of 3-93 mg/kg in grain for human consumption (Canady et al., 2001).

Mycotoxin contamination of feed ingredients also has been a serious threat to livestock industry, particularly swine production. Typical acute poisoning symptoms of DON to livestock animals include weight loss, feed refusal, nausea, vomiting, and bloody diarrhea (Rotter et al., 1996; Pestka and Smolinski, 2005; Pestka, 2007). Contamination of grains with DON creates a food safety risk, a serious threat to the livestock industry, and a negative impact on international trade (Wu, 2004; Wu, 2006; Kendra and Dyer, 2007).

The current practice to reduce or contain mycotoxin contamination is focused mainly on the prevention of contaminated grain materials from entering the food chain through regulation, detection and compliance. Also, various physical and chemical decontamination techniques have been developed to reduce the concentration of DON in affected grains. For example, cleaning methods, such as gravity and sieving separation, dehulling and washing procedures can reduce the concentration of DON in wheat and maize (Trenholm et al., 1992). Thermal treatments by microwave or convection also may be used (Young, 1986). Chemical detoxification by using oxidants such as ozone (Young, 1986; Young et al., 2006), reducing reagents such as ascorbic acid, sodium bisulfite (NaHSO₃) and sodium metabisulfite (Na₂S₂O₅) (Swanson et al., 1984; Young et al., 1986b; Dänicke et al., 2005), and alkali such as sodium hydroxide (Young et al., 1986a) have been investigated. However, these techniques have several disadvantages, including inefficiency, residues of harmful chemicals, significant losses in nutritive value, and losses in palatability of detoxified food or feed (Karlovsky, 1999).

A variety of approaches have been used to reduce effects of mycotoxins on livestock industries. The use of adsorbents as feed additives is common. Such adsorbents may include alfalfa fiber, activated carbon, hydrated sodium calcium aluminosilicate (HSCAS), zeolite, organozeolite, sepiolite, clinoptilolite, bentonite, esterified glucomannan (Galvano et al., 1998; Lemke et al., 2001; Diaz et al., 2002; Tomasevic-Canovic et al., 2003). Unfortunately, the use of adsorbents in feeds to remove mycotoxins is not only relatively expensive, but also specific to particular mycotoxins. Some of the most popularly used adsorbents, such as HSCAS and sepiolite, are not effective against DON (Galvano et al., 1998). The use of grains with no or low mycotoxin contamination to dilute mycotoxin level in feed is another common approach. Furthermore, the dilution method to reduce mycotoxin contamination by mixing contaminated ingredients with high quality grains is difficult to implement because the degree of contamination is often not known and thus there are questions about the extent of dilution needed to reach contamination levels which would be considered acceptable. Some European countries have already banned the use of this procedure (Commission Regulation (EC), 2001).

Despite a plethora of information regarding the biochemistry, toxicity, and modes of action of mycotoxins, there still remain no viable solutions for either pre- or post-harvest control/eradication of these toxins (Cardwell et al., 2001). In developed countries, substantial costs are incurred through testing, compliance and research to prevent entrance of mycotoxins into the food chain. In the United States these costs are estimated to be about US$500 million to $1.5 billion per year (CAST (Council for Agricultural Science and Technology), 1989). In Canada, losses of $100 million were accrued in 1996 following a Fusarium epidemic in Ontario (Schaafsma, 2002).

There is a need in the art for novel products and methods for mycotoxin control and/or decontamination. Furthermore, there is a need in the art for specific, efficient and environmentally sound ways for decontamination/detoxification of mycotoxins.

### SUMMARY OF THE INVENTION

The present invention relates to detoxification microorganisms. More specifically, the present invention relates to bacterial isolates and methods for detoxifying mycotoxins.

According to the present invention there is provided a bacterial isolate defined by accession number 040408-1 filed with the International Depository Authority of Canada.

Also provided by the present invention is a composition comprising bacteria as defined above.

The present invention also contemplates a composition as defined above, wherein the composition comprises a carrier.

Also provided by the present invention is a composition as defined above, wherein the carrier is a food or food product contaminated or susceptible to contamination by trichothecene mycotoxins or organisms that produce trichothecene mycotoxins.

The present invention also contemplates a composition as defined above wherein the trichothecene mycotoxins comprise DON.

Also provided by the present invention is a method of preventing or reducing mycotoxin contamination in a food or food product by treating the food or food product with bacteria as defined above.

The present invention also contemplates a method as defined above, wherein the mycotoxin contamination comprises trichothecene mycotoxins, preferably DON.

The present invention also provides a kit comprising bacteria as defined above and one or more of the following:

a) one or more carriers for holding, suspending, diluting, adhering, enveloping, culturing, growing, or freezing/cryopreserving the bacteria;

b) one or more devices for combining or formulating the bacteria with the one or more carriers of a);

c) one or more devices for treating a food or food product with the bacteria or a composition comprising the bacteria, and;

d) instructions for growing the bacteria, formulating the bacteria with the one or more carriers, using one or more devices for treating a food or food product, or a combination thereof

The present invention also provides a method of screening for microorganisms that are capable of reducing DON comprising,

a) obtaining a soil sample;

b) culturing bacteria in the soil sample under conditions to enrich for bacteria that are capable of reducing DON;

c) isolating one or more single colonies of bacteria from the step of culturing (step b), and;

d) individually testing the one or more single colonies in an assay to confirm if the colony or colonies are capable of reducing DON.

The present invention also contemplates a method as defined above further comprising culturing, purifying, isolating or any combination thereof the one or more single colonies that are capable of reducing DON. In still a further embodiment step b) may be preceded by a step of extracting bacteria from the soil sample.

This summary of the invention does not necessarily describe all features of the invention.

### BRIEF DESCRIPTION OF THE DRAWINGS

These and other features of the invention will become more apparent from the following description in which reference is made to the appended drawings wherein:

FIG. 1 graphically shows a time course of DON reduction and transformation product formation by bacterial strain 040408-1 in CMB medium at 28° C.

FIG. 2 graphically shows the effect of shaking culture conditions on the growth and DON-reduction activity of bacterial strain 040408-1 in CMB medium at 28° C.

FIG. 3 graphically shows the effect of DON on the growth of bacterial strain 040408-1 in CMB medium at 28° C.

FIG. 4 graphically shows the effect of DON on the DON-reduction activity of bacterial strain 040408-1 in CMB medium at 28° C.

FIG. 5 graphically shows the effect of temperature on the DON-reduction activity of bacterial strain 040408-1 in CMB medium.

FIG. 6 graphically shows the effect of inoculation concentration on the DON-reduction activity of bacterial strain 040408-1 in CMB medium at 28° C.

FIG. 7 shows HPLC chromatograms of the extracts of DON transformation in MM medium by soils #11, 17, 21, 31, 110-1, and 165-2.

FIG. 8 shows HPLC chromatograms of the extracts of DON transformation in CMB medium by soils #11, 17, 21, 31, 110-1 and 165-2.

FIG. 9 shows HPLC chromatograms of the extracts of DON transformation in MM, MMY, MMP, MMPT, CMB, and CMBPD media by soil # 165-2.

FIG. 10 shows the effect of DON, 3-epi-DON and 3-keto-DON on cell viability of Caco-2 cells at various concentrations. The values are expressed as present of control response and each value is a result of four experiments with six replicates each.

FIG. 11 shows the effect of DON, 3-epi-DON and 3-keto-DON on DNA synthesis in 3T3 mouse fibroblasts at various concentrations. The values are expressed as present of control response and each value is a result of four experiments with six replicates each.

FIG. 12 shows the effect of supplementation of minerals on growth (A) and deoxynivalenol transforming ability (B) of the bacterial isolate 040408-1 in different media. Values were determined after 72 h in shaken culture (200 rpm) at 28° C. Values in the same pair of columns with different superscripts differ significantly according to Paired T test (P<0.05). CMB is shown as a reference control.

FIG. 13 shows reaction metabolites from deoxynivalenol biotransformation in different media. Values were determined after 72 h in shaken culture (200 rpm) at 28° C. Stacked columns display cumulative totals of DON biotransformation products for CSL, PEP, YEA and CMB only. DON stereoisomer (3-epi-DON) values differ significantly according to Tukey's multiple range test (P<0.05).

FIG. 14 shows biotransformation cures for DON and metabolites. Values were determined after 48 h in shaken culture (200 rpm) at 28° C. Samples were collected every 12 h. DON and metabolites were quantified on the basis of integrated peak areas. It was assumed that the molar response factor for each metabolite was equal to that of DON. DON stereoisomer indicates 3-epi-DON.

### DETAILED DESCRIPTION

The following description is of a preferred embodiment.

Provided herein is the isolation and identification of microorganisms capable of detoxifying trichothecene mycotoxins to one or more less toxic products, for example, but not limited to, detoxification of DON to one or more less toxic products. Detoxification of trichothecene mycotoxins such as DON may occur by one or more routes, for example, but not wishing to be limiting or bound by theory, epimerization of deoxynivalenol to epi-deoxynivalenol, or other routes.

According to the present invention there is provided a bacterial isolate defined by accession number 040408-01 filed with the International Depository Authority of Canada (IDAC) on Apr. 4, 2008. Bacteria from the isolate exhibit mycotoxin detoxifying activity, for example, but not limited to DON detoxification activity. As will be evident from the information provided herein, the bacterial isolate comprises bacteria removed from their natural surrounding. Preferably, the bacterial isolate does not comprise soil particles. More preferably the isolate is substantially pure meaning that it does not comprise other microorganisms in the isolate.

The present invention also provides a composition comprising bacteria as defined by IDAC accession number 040408-01 and a carrier. By the term “carrier” it is meant a liquid, solid, liquid-solid or semi-solid substrate or medium for holding/retaining, suspending, diluting, adhering, enveloping, culturing, growing, freezing/cryopreserving or any combination thereof, the bacteria as defined above. For example, but not to be considered limiting in any manner, the carrier may comprise a culture medium, such as, without limitation, minimal medium; minimal medium supplemented with one or more additives, for example, but not limited to yeast extract, peptone, tryptone or a combination thereof; corn meal broth with or without additives such as, without limitation, salts, peptone, dextrose or other sugars; corn meal agar; rice medium or any combination thereof. Other carriers including, but not limited to culture media and the like as would be evident to a person of skill in the art are also meant to be encompassed by the term “carrier” as used herein. In a preferred embodiment, the carrier does not substantially affect the detoxification ability of the bacteria in association therewith.

It is also contemplated that the carrier also may comprise a food, food product or a combination of food or food products. By the term “food or food products” it is meant any food, feed or combination of foods and feeds, either in natural, harvested or processed form for human and/or animal consumption. Any food or food product that comprises trichothecene mycotoxins, that is capable of being contaminated by trichothecene mycotoxins or that is susceptible to infection by microorganisms producing trichothecene mycotoxins is specifically included as food or food products herein. Representative examples of foods or food products include without limitation cereals for example, but not limited to corn, barley, rice, wheat, oats, sorghum, rye or mixtures thereof Accordingly, there is provided a composition comprising bacteria as defined by accession number 040408-01 and a carrier, wherein the carrier is food or food product, for example a human or animal food or feed product. In a preferred embodiment the food or food product is contaminated or susceptible to contamination by DON or microorganisms that are capable of producing DON. Similarly, there is contemplated a food or food product that comprises bacteria defined by accession number 040408-01 or that is treated to comprise the bacteria as defined by accession number 040408-01.

The present invention also provides a method of reducing mycotoxin contamination in a food or food product by treating the food or food product with bacteria as defined by accession number 040408-01 or a composition comprising bacteria as defined by accession number 040408-01. In a preferred embodiment, the mycotoxins comprise trichothecene mycotoxins, more preferably DON.

The present invention also provides a method of preventing mycotoxin contamination in a food or food product by treating the food or food product with bacteria as defined by accession number 040408-01 or a composition comprising bacteria as defined by accession number 040408-1.

Also contemplated by the present invention is a kit comprising bacteria as defined by accession number 040408-01 and one or more of the following:
a) one or more carriers;
b) one or more devices for combining or formulating the bacteria with the one or more carriers of a);
c) one or more devices for treating a food or food product with the bacteria or a composition comprising the bacteria as described above, and;
d) instructions for growing the bacteria, formulating the bacteria with the one or more carriers, using one or more devices for treating a food or food product, or a combination thereof.

It is to be understood that the bacteria as defined above may be combined with the one or more carriers as defined in a) or the two may be separate. Also possible is a kit that comprises bacteria, bacteria and carrier, and carrier as three separate components.

In a further embodiment of the present invention, there is provided a method of screening for microorganisms that are capable of reducing DON comprising,
a) obtaining a soil sample;
b) culturing bacteria in the soil sample under conditions to enrich for bacteria that are capable of reducing DON;
c) isolating one or more single colonies of bacteria from the step of culturing (step b);
d) individually testing the one or more single colonies in an assay to confirm if the colony or colonies are capable of reducing DON;
and optionally;
e) culturing, purifying, isolating or any combination thereof one or more single colonies that are capable of reducing DON.

In the method as defined above, step b) may be optionally preceded by a step of extracting bacteria from the soil sample with water, other medium, or the like, prior to culturing the bacteria under conditions that result in enrichment in bacteria that reduce DON.

In a preferred embodiment of the method as defined above, the soil sample or extracted bacteria derived from the soil sample is cultured with a ground food crop comprising DON or a ground food crop comprising DON and a microorganism capable of producing DON such as, but not limited to F. graminearum. In a more preferred embodiment, the enrichment step is performed by culturing the bacteria for about 6 weeks in an aerobic environment at a temperature of about 28° C. Other conditions also may be employed as would be evident to a person of skill in the art.

The present invention will be further illustrated in the following examples.

### EXAMPLES

### Example 1

Chemicals, Cultural Media, Microorganisms and Soils

Deoxynivalenol (DON or vomitoxin) standard, glucose, sucrose, dextrose, xylose, (NH₄)₂SO₄, (NH₄)₂HPO₄, K₂HPO₄, KH₂PO₄, MgSO₄, K₂SO₄, FeSO₄, MnSO₄, carboxymethyl cellulose (CMC), NH₄NO₃.7H₂O, Dulbecco's modified eagle medium (DMEM), fetal calf serum (FCS), penicillin, streptomycin, sodium pyruvate, phosphate buffered saline (PBS), trypsin, ethylenediamine tetraacetic acid (EDTA), thiazolyl blue tetrazolium bromide (MTT) and dimethyl sulfoxide (DMSO) were purchased from Sigma-Aldrich (Oakville, Canada). DON used in the biotransformation assays was purified from F. graminearum rice culture using high speed counter current chromatography (He et al., 2007). Standard 3-keto-DON and mouldy corn were obtained from the Eastern Cereal and Oilseed Research Centre, AAFC, Ottawa, ON, Canada. HPLC grade methanol was obtained from Caledon Labs, (Georgetown, Canada). DIFCO potato dextrose agar (PDA), DIFCO tryptic soy broth (TSB), DIFCO Lauria Bertani broth (LBB), DIFCO malt extract broth (MEB), DIFCO nutrient broth (NB), DIFCO peptone, DIFCO tryptone, and DIFCO yeast extract were purchased from Fisher Scientific (Ottawa, ON, Canada).

Minimal medium (MM): 1 L medium contained 10.0 g sucrose, 2.5 g K₂HPO₄, 2.5 g KH₂PO₄, 1.0 g (NH₄)₂HPO₄, 0.2 g MgSO₄.7H₂O, 0.01 g FeSO₄, and 0.007 g MnSO₄. MM+yeast medium (MMY): MM medium with 0.5% yeast extract. MM+peptone medium (MMP): MM medium with 1% peptone. MM+peptone+tryptone medium (MMPT): MM medium with 1% peptone and 1% tryptone. Corn meal broth without salts (CMB/WO/S): 40 g corn meal soaked in 1 L water at 58° C. for 4 h, allowed to stand for 2 h, and was then filtered through a Whatman No. 1 filter paper (Whatman; Maidstone, Kent, UK). Corn meal broth (CMB): One liter of CMB/WO/S was added 3 g (NH₄)₂SO₄, 1 g K₂HPO₄, 0.5 MgSO₄, 0.5 K₂SO₄, 0.01 g FeSO₄, 0.007 g MnSO₄, and 5 g yeast extract. Corn meal broth+peptone+dextrose medium (CMBPD): 2% peptone and 2% dextrose was added to CMB. Corn meal agar (CMA): CMB supplemented with agar to a final concentration of 1.5%. Mouldy corn meal broth (MCMB): 40 g mouldy corn meal soaked in 1 L water at 58° C. for 4 h, allowed to stand for 2 h, and was then filtered through a Whatman No. 1 filter paper (Whatman; Maidstone, Kent, UK); one liter of this filtrate was added 3 g (NH₄)₂SO₄, 1 g K₂HPO₄, 0.5 MgSO₄, 0.5 K₂SO₄, 0.01 g FeSO₄, 0.007 g MnSO₄, and 5 g yeast extract. Rice medium (RM): 40 g rice powder soaked in 1 L water at 58° C. for 4 h, allowed to stand for 2 h, and then filtered through a Whatman No. 1 filter paper (Whatman; Maidstone, Kent, UK). Yeast+glucose: 1 L medium containing 5.0 g yeast and 10.0 g glucose. BYE: 1 L medium containing 0.5 g of NH₄NO₃, 0.2 g of yeast extract, 50 mg of H₃BO₄, 40 mg of MnSO₄.4H₂O, 20 mg of (NH₄)₆Mo₇O₂₄, 4 mg of CuSO₄.5H₂O, and 4 mg of CoC1₆.6H₂O and 5 mM potassium phosphate buffer (adjusted to pH 7.0 with NaOH) (Shima et al., 1997).

F. graminearum, isolate 178148, was obtained from the Canadian Collection of Fungal Cultures (Ottawa, ON, Canada). The fungus was grown on PDA for 5-7 d at 23° C. in an Innova 4230 incubator (New Brunswick Scientifica, Edison, N.J., USA) before being used.

Samples of one hundred and sixty five agricultural soils were collected from fields previously cropped to corn, wheat, barley, alfalfa, grass, soybean, pea, potato, clover, pumpkin, tobacco, ginseng, apple, peach, or rape crops. Soil (1-2 L, including crop debris) from the top layer (0-30 cm) was randomly collected. Crop debris was blended into small pieces (less than 2 mm) using a Waring laboratory blender (Fisher Scientific) at high speed for 1-2 min, and then mixed with the individual soil samples. Soil samples were stored at 4° C.

Isolation of DON-Reducing Microorganisms by Enrichment for Bacteria in Agricultural Soils

Mouldy corn kernels contaminated with 95 μg DON/g were mixed and ground in a Waring laboratory blender (Fisher Scientific, Ottawa, ON, Canada) at high speed for 1-2 min. The corn powder was autoclaved at 121° C. for 30 min. Macroconidia of F. graminearum were prepared by using CMC medium (He et al., 2007). A sample of each agricultural soil (0.5 L) was mixed with above mouldy corn powder (100 g) and F. graminearum suspension (5 mL of 1×10⁴ macroconidia/mL). The soil mixture was incubated at 28° C. and 80% relative humidity for 6 weeks. Total fifty-seven soils were enhanced with F. graminearum-mouldy corn: fifty-five soils collected in April-May 2006, one mixture of soils collected in October-November of 2004, and one mixture of all the soils collected in 2004 and 2006. Soil, soil treated with mouldy corn, soil treated with F. graminearum, autoclaved soil treated with mouldy corn and F. graminearum served as blank control, nutrient control, pathogen control and non-soil-microorganism control, respectively.

One hundred and sixty-five agricultural soils and fifty seven treated soils were screened for the ability to reduce DON. Sterile deionized water was added to soil (1:1, v/v) and the mixture was shaken at 200 rpm at room temperature (23° C.) for 4 h. A 100 μL aliquot was immediately collected from the resulting soil suspension and treated with 100 μL 1000 μg/mL DON in sterile water and 800 μL MM medium. Cultures were grown at 28° C. on a rotary shaker at 200 rpm. After incubation for 72 h, the above cultures were extracted and analyzed by HPLC as described herein.

DON-reducing activities were examined as follows: The DON-reducing soil cultures were sub-cultured in the same medium in which the DON- reducing activities were detected. Replacement of the culture with sterile water served as a blank control; an autoclaved soil suspension served as a physical absorption control; and a soil suspension filtered through a 0.22 μm mixed esters cellulose (MEC) sterile syringe filter (Fisher) served as a chemical reaction control. These controls were prepared for comparison with soil samples that had DON- reducing activities.

Different media were tested for enhanced DON-reducing activity. The DON-reducing soils were sub-cultured in MM, MMY, MMP, MMPT, CMB, and CMBPD media at 28° C. for 72 h under aerobic condition at 28° C. on a rotary shaker at 200 rpm for 72 h and also under anaerobic conditions (5% H₂ and 10% CO₂ balanced N₂) at 23° C. for 72 h with hand-mixing every 6 h, respectively. To each individual soil sample, replacement of the soil suspension with sterile water served as a blank control; an autoclaved soil suspension served as a physical absorption control; and a soil suspension filtered through a 0.22 μm MEC sterile syringe filter (Fisher) served as chemical reaction control.

The DON-reducing soil cultures from above were serially diluted up to 10⁻¹⁰ using CMB medium. Two parameters were examined; one was DON-reducing activity and the other was the population of microorganisms. For tests of DON-reducing activity, 100 μL solution from the serial was sub-cultured with DON (100 gL of 1000 μg/mL DON standard) in 800 ₜit CMB at 28° C. on a rotary shaker at 200 rpm for 72 h. Cultures were analyzed as described below. For tests of population of microorganisms, to each dilution, 100 μL solution from each serial dilution was plated on an CMA plate and incubated at 28° C. After 48-72 h incubation, the colony-forming units (CFU) were counted. These two results were combined for each serial dilution. The serial dilution showing the lowest population of microorganisms exhibiting DON reducing activity was identified and another serial dilution was made. The culture was sub-cultured and tested for DON-reducing activity as above.

The same procedure was repeated 8 times. Single colonies were picked from the agar plate corresponding to the highest dilution that still had activity of DON reduction. These colonies were sub-cultured in CMB and their activities of DON reduction were evaluated using the methods as described below.

Extraction of DON from Culture and High Performance Liquid Chromatography (HPLC) Analysis

To each 500 μL culture, 500 μL methanol was added. The mixture was allowed to stand for 2 h and filtered through a 0.45 μm polyvinylidine fluoride (PVDF) syringe filter (Whatman; Maidstone, Kent, UK) before being analyzed by HPLC. Identification and quantification of DON were achieved using an Agilent Technologies 1100 Series HPLC system with a Luna C18 (2) column (150×4.6 mm, 5 μm) (Phenomenex, Torrance, Calif., USA). The binary mobile phase consisted of solvent A (methanol) and solvent B (water) and the gradient program began at 22% A, increased linearly to 41% A at 5 min, 100% A at 7 min, held 100% A from 7 to 9 min, and returned to 22% A at 11 min. There was a 2 min post-run under starting conditions for re-conditioning. The flow rate was 1.0 mL/min and the detector was set at 218 nm. Identification of DON was achieved by comparing its retention time and UV-Vis spectra with those of a DON standard. Quantification was based on reference to a calibration curve of DON standard (He et al., 2007)

Liquid Chromatography-Mass Spectrometry (LC-MS) and Nuclear Magnetic Resonance (NMR) Identification of DON Transformation Products

LC-MS was performed using HPLC with a Phenomenex Luna C18 (2) column (150×4.6 mm, 5 μm) coupled to a photodiode array UV detector (Finnigan MAT Spectra System UV6000LP; San Jose, Calif., USA) equipped with a Finnigan LCQ Deca atmospheric pressure chemical ionization (LC-APCI-MS) operated in the positive ion mode. Detailed instrumental parameters were described before (He et al., 2007). The major product of DON transformation by bacterial strain 040408-1 was purified from the DON transformation culture using high speed countercurrent chromatography. Proton NMR spectra of DON and Compound-1 were recorded in DMSO-d₆ using Bruker Avance-400 and -600 spectrometers (Bruker BioSpin Ltd., Milton, ON, Canada). Also, H—H correlation spectroscopy (COSY) and heteronuclear single quantum coherence (HSQC) were also recorded. The parallel nuclear overhauser effect (NOE) tests of DON and compound 1 were conducted using a Bruker Avance-400 spectrometer (Bruker BioSpin Ltd., Milton, ON, Canada).

Identification of Bacterial Strain 040408-1 (Deposit 040408-1)

Bacterial identification was performed by MIDI® gas chromatographic analysis of fatty acids methyl esters (GC-FAME), Biolog bacterial identification and 16S rRNA gene sequencing method. Morphological characterization by scanning electron microscope (SEM) was done in the electron microscope lab of the department of Food Science, and transmission electron microscope (TEM) was performed in the Guelph Regional Integrated Imaging Facility (GRIIF), Transmission Electron Microscope Facility, department of Molecular and Cell Biology, University of Guelph.

Characterization of bacterial strain 040408-1 for its activities of DON transformation: The effect of culture conditions on DON reduction by bacterial strain 040408-1.

CMB medium (10.0 mL) was inoculated with a loop of bacterial strain 040408-1 culture (1 μL). The culture was incubated at 28° C. for 72 h with shaking at 200 rpm.

The culture was adjusted to a cell concentration of using CMB medium based on a calibration curve (a standard curve of optical density (O.D.) vs. cell number, λ=600 nm).

To test the effect of aerobic/anaerobic condition, shaking and culture media on the growth and the activity of DON reduction by bacterial strain 040408-1, each 100 μL bacterial strain 040408-1 culture having a cell concentration of 1×10⁶ CFU/mL was added to 100 μL of 1000 μg/mL DON and 800 μL MM, MMY, MMP, MMPT, CMB, CMBPD, BYE, rice medium, malt extract, corn meal broth without salts (CMB/WO/S), nutrient broth, TSB, Lauria Bertani and Yeast+glucose media. Cultures were incubated at 28° C. for 72 h under aerobic condition at 28° C. on a rotary shaker at 200 rpm, and also under anaerobic conditions (5% H₂ and 10% CO₂ balance N₂) at 23° C. with hand-mixing approximately every 6 h, respectively.

To test the effect of temperature on the growth and the activity of DON reduction by bacterial strain 040408-1, cultures containing bacterial strain 040408-1 1×10⁵ CFU/mL, 100 μg/mL DON and CMB medium were incubated at 4, 15, 20, 28, 37° C. on a rotary shaker at 200 rpm.

To test the effect of inoculation concentration on the growth and the activity of DON reduction by bacterial strain 040408-1, cultures containing bacterial strain 040408-1 1×10⁰, 1×10¹, 1×10², 1×10³, 1×10⁴, 1×10⁵, 1×10⁶, 1×10⁷, 1×10⁸, 5×10⁹ CFU/mL, 100 μg/mL DON and CMB medium were incubated at 28° C. on a rotary shaker at 200 rpm for 72 h. After 72 h incubation, they were extracted and analyzed as previously described.

The Effect of DON on the Growth and Function of Bacterial Strain 040408-1

CMB medium (12.0 mL) was added with 1.5 mL bacterial strain 040408-1 culture of 1×10⁶ CFU/mL and 1.5 mL DON standard (DON in sterile water, 1000-40000 μg/mL). The culture was incubated at 28° C. on a rotary shaker at 200 rpm for up to 132 h.

To evaluate the effect of DON on the growth of bacterial strain 040408-1, the cell number of bacterial strain 040408-1 was counted at every 12 h. Each time, 100 μL it culture was made in serial dilutions with CMB medium. Each of the dilutions (100 μL) was streaked on corn meal agar plates and the CFUs were counted after incubation at 28° C. for 72-96 h.

To evaluate the effect of DON on the function of bacterial strain 040408-1, 150 μL culture was removed at 6, 12, 24, 36, 48, 60, 72, 84, 96, 108, 120, 132 h, and then added to 150 μL methanol. The mixture was allowed to stand for 2 h and centrifuged at 18000 g for 5 min (Micromax® microcentrifuge, Milford, Mass., USA) before being analyzed by HPLC.

Statistical Analysis of DON Reduction

Each sample was analyzed in triplicate and the means were determined. Relevant reductions of DON were calculated as follows: DON reduction (%)=(C_{DON added}−C_{DON residual})/C_{DON added}×100. All data were analyzed using SAS (SAS for Windows, Version 9.1, SAS institute, Cary, N.C., USA). A type I error rate of 0.05 was used for all analyses. Treatments were arranged in a completely randomized design. Differences among treatments were determined using a protected least significant difference (PLSD) test.

Toxicity of Transformation Products of DON: Cell Culture

Human colonic carcinoma Caco-2 cells (ATCC No. HTB-37) and Swiss mouse fibroblast NIH/3T3 cells (ATCC No. CRL-1658) were obtained from the American Type Culture Collection (ATCC). Cells were grown to confluence in Dulbecco's modified eagle medium (DMEM) medium containing 4.5 g/L glucose, 10% (v/v) fetal bovine serum, penicillin (100 IU/ml) and streptomycin (100 μg/ml) in a humidified incubator at 37° C. in an atmosphere of 95% air and 5% CO₂. Cells were sub-cultured weekly. The passes of 25-35 and 14-23 for Caco-2 and 3T3 cells were used, respectively. The cells were then trypsinized, diluted, added to 96-well plastic culture plates (Corning Costar®, Sigma) and incubated in DMEM containing test chemicals.

Test of Metabolic Activity by MTT Bioassay

MTT test was applied to assess cell viability on the base of the capability of viable cells to convert soluble MTT (yellow) to purple formazan crystals. This dehydroxylation is catalyzed by enzymes in the mitochondria. Cells were incubated in a humidified incubator at 37° C. in an atmosphere of 95% air and 5% CO₂. Caco-2 cells were pre-seeded 24 h in 96 culture plates with a density of 35,000 cells/cm² (0.32 cm²/well) by adding 100 μL 1.1×10⁵ cells/mL cell suspension in DMEM medium, and then DON, 3-epi-DON and 3-keto-DON in 100 μL fresh DMEM medium were added to wells. Final concentrations ranged from 0.0100-5.00 μg/mL for DON, 1.00-1000 μg/mL for 3-epi-DON and 0.0100-10.0 μg/mL for 3-keto-DON. MTT was dissolved in PBS to make a 5 mg/mL solution, and the resulting solution was filtered through a 0.22 μm MEC sterile syringe filter (Fisher). After 48 h incubation, 25 μL MTT solution was added to each well of 96 well culture plates and incubated for additional 4 h. At the end of incubation, medium was removed, and 200 μL DMSO was added to extract the formazan. After stirring for 1 min, the absorbance was determined at 570 nm using a Powerware™ XS, Universal Microplate Spectrophotometer (BIO-TEK® Instruments Inc., Winooski, Vt., USA) (Cetin and Bullerman, 2005; Kouadio et al., 2005; Sergent et al., 2006).

Test of DNA Synthesis Activity by a Cell Proliferation ELISA Employing BrdU Incorporation

DNA synthesis was measured by immunoassay on the basis of the incorporation of BrdU during DNA synthesis. The procedure followed the instruction manual of the cell proliferation ELISA, BrdU (colorimetric) kit (Cat. No. 1164229001, Roche Diagnostics, Laval, Quebec, Canada). 3T3 cells were pre-seeded 24 h in 96 culture plates with a density of 31,000 cells/cm² (0.32 cm²/well) by adding 100 μL 1.0×10⁵ cells/mL cell suspension in DMEM medium at 37° C. in an atmosphere of 95% air and 5% CO₂, and then DON, 3-epi-DON and 3-keto-DON in 100 μL fresh DMEM medium were added into wells. Final concentrations ranged from 0.0100-5.00 μg/mL for DON, 1.00-1000 μg/mL for 3-epi-DON and 0.0100-10.0 μg/mL for 3-keto-DON. After 24 h incubation, 20 μL 100 μmol/L BrdU labeling solution was added to each well and the culture was reincubated at 37° C. for additional 12 h. The pyrimidine analogue BrdU replaces thymidine in the DNA of reproducing cells in this labeling period. At the end of the incubation, medium was removed. Cells were fixed and the DNA was denatured by adding 200 μL/well FixDenat and incubating at room temperature (23° C.) for 30 min. Following removal of removed FixDenat, 100 μL/well antibody anti-BrdU-POD working solution was added to let the BrdU incorporate in newly synthesized cellular DNA. This incubation period was 90 min at room temperature. After removal of the reaction solution, the culture was washed with PBS 3 times, and 100 μL substrate solution was added and incubated at room temperature for another 30 min. The absorbance was determined at 370 nm with a reference wavelength of 492 nm using a Powerware™ XS, Universal Microplate Spectrophotometer (BIO-TEK® Instruments Inc., Winooski, Vt., USA). Absorbance values correlate to the amount of DNA synthesis, and thereby to the proliferation of cells (Eriksen et al., 2004).

Mean values of the absorbance of the six replicate samples at each concentration of test chemicals were compared to the mean value of the corresponding control. Dose response curves were computer plotted (Using Sigma Plot 10.0, Systat Software Inc., San Jose, Calif., USA) , and IC50 values were calculated by computer program (ProStat 4.12, Poly Software Inc., Pearl River, N.Y., USA). Data are presented as the mean of 4 independent experiments.

Test of DON-Reduction Activity of Bacterial Strain 040408-1 in Mouldy Corn Media with Different Stickiness

To test the growth and function of bacterial strain 040408-1 in liquid culture conditions, CMB and MCMB were used. The incubations were performed under aerobic conditions at 28° C. with shaking at 200 rpm for 72 h. CMB medium (5 mL) containing 1×10⁵ CFU/mL bacterial strain 040408-1 served as control; CMB medium (5 mL) containing 5×10⁴ CFU/mL F. graminearum macroconidia served as F. graminearum-control. Treatments were MCMB medium (5 mL) containing either bacterial strain 040408-1 or F. graminearum macroconidia, or both whose concentrations were same as above controls.

To test the capability of DON reduction of bacterial strain 040408-1 in suspension medium, a mixture comprising mouldy corn and bacterial strain 040408-1 in fresh CMB medium (1:9, m/v) was tested. Mouldy corn powder (2.5 g, 1.3 mg DON/g) was soaked in 20 mL fresh CMB medium for 12 h before being added to 2.5 mL of bacterial strain 040408-1 culture (1×10⁹ CFU/mL). The culture was then incubated under aerobic conditions at 28° C. with shaking at 200 rpm for 72 h.

To test the capability of DON reduction of bacterial strain 040408-1 in paste medium, 2.5 g mouldy corn powder (1.3 mg DON/g) was incubated with 2.5 mL 1×10⁸ CFU/mL bacterial strain 040408-1 culture under aerobic condition at 28° C. with shaking at 200 rpm for 72 h.

Test of Reduction of Other Trichothecenes by Bacterial Strain 040408-1

Bacterial strain 040408-1 (1×10⁵ CFU/mL) was cultured in CMB medium containing 100 μg/mL 3-acetyl-DON, 15-acetyl-DON, T-2 toxin, HT-2 toxin and Roridin A at 28° C. with shaking at 200 rpm for 72 h. Cultures were extracted as described herein and analyzed using LC-MS (Finnigan MAT Spectra System UV6000LP). A Zorbax Eclipse XDB-C18 column (150×4.6 mm, 3.5 μm) was used. The binary mobile phase consisted of solvent A (methanol) and solvent B (water) and the gradient program began at 25% A, increased linearly to 75% A at 15 min, 80% A at 20 min, held 80% A from 20 to 23 min, and returned to 25% A at 26 min. There was a 3 min post-run under starting conditions for re-conditioning. The flow rate was 1.0 mL/min and the photodiode array UV detector was set at 218 nm.

### RESULTS

Reduction of DON by Enriched Soils

One hundred and sixty-five agricultural soils were screened for the ability to reduce DON concentration when DON was artificially added to soil suspensions. None of the soil samples tested showed a significant reduction in DON without enrichment (data not shown). However, after enrichment, thirty-three of the fifty-seven soils demonstrated the ability to reduce DON by at least 10% (PLSD_{(0.05)}=9.4%) (data not shown). Soils #17, 31, 110-1 and 165-2 were efficient soils, and reduced DON by more than 50%. These soils were selected for further study. Soils #11 and 21 reduced DON by only 10%, they were also chosen for further study because these soils were collected from wheat and corn fields, respectively, where Fusarium head blight and Gibberella ear rot have often occurred. Sources of these soil samples are shown in Table 1. When tested under aerobic conditions, the soil suspensions in MM medium reduced DON by about 10˜73%. However, the autoclaved suspensions (physical controls) and the filtered suspensions (chemical controls) did not reduce DON in the culture. These results suggest that the reduction of DON is due to biological activities (Table 1).

The suspensions made from the six selected soils reduced DON in one or more of MM, MMY, MMP, MMPT, CMB, and CMBPD media under aerobic conditions. DON reduction was not observed under anaerobic conditions in any of the above six media (data not shown). CMB medium was an efficient test medium tested and the DON recoveries of these six selected soils were about 0-17.6% in this medium. Therefore, CMB medium was chosen as the screening medium for DON-reducing microorganisms.

Soil enrichment of Soil #17 was repeated once (Table 2). DON was not reduced by either the autoclaved suspension of Soil #17 enhanced with F. graminearum+corn or the cell-free filtrate of Soil #17 enhanced with F. graminearum+corn. Only the soil suspensions containing living microorganisms transformed DON into different products, which suggested that the reduction of DON was due to microbial activity. DON reduction was detected in the treatments with Soil #17 enhanced with F. graminearum+corn and Soil #17 enhanced with corn. F. graminearum produced DON (23.1±11.2 μg/g) in autoclaved Soil #17 when incubated with corn. Other results indicated that DON recovery from Soil #17 enhanced with corn and F. graminearum in CMB medium (5.2 μg/mL, Table 2) was much lower than that in MM medium (53.2 μg/mL, Table 1). This also suggested that CMB medium was suitable for use in screening for DON-reducing microorganisms.

Isolation of Bacterial Strain 040408-1, a DON Reducing Microorganism from Soils

A white tiny colony was selected after eight times of concurrent purification on the base of nutrient selection and stepwise decontamination. Its source was a treated soil that was originally collected from an alfalfa field in 2006. The resulting 16S rRNA gene from bacterial strain 040408-1 had 97-94% sequence similarity to certain strains (Table 10).

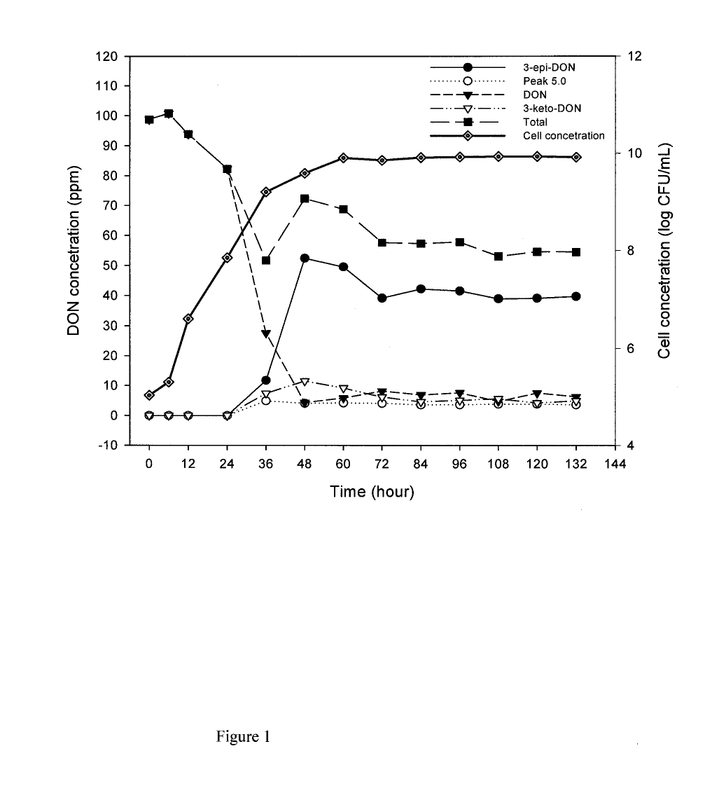

It converted DON to one major product (3-epi-DON) and two minor products (Peak 5.0 and 3-keto-DON). The production of 3-epi-DON and the disappearance of DON were coincident (see FIG. 1).

Physiological Characterization of Bacterial Strain 040408-1 for DON Reduction

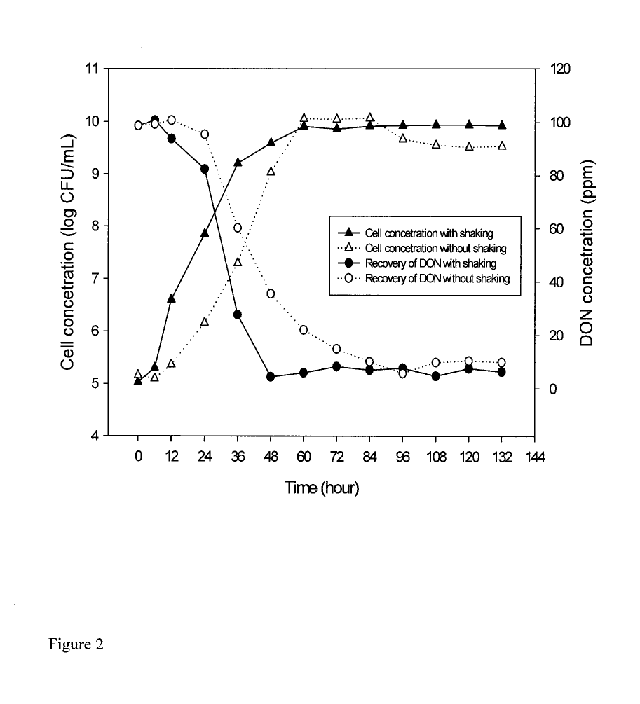

During incubation in CMB at 28° C. without shaking, the growth and function of bacterial strain 040408-1 were not inhibited, but delayed by 12-24 h compared to those with shaking at 200 rpm (FIG. 2). The coincidence of the growth of bacterial strain 040408-1 and the reduction of DON as previously described (FIG. 1) was observed as well.

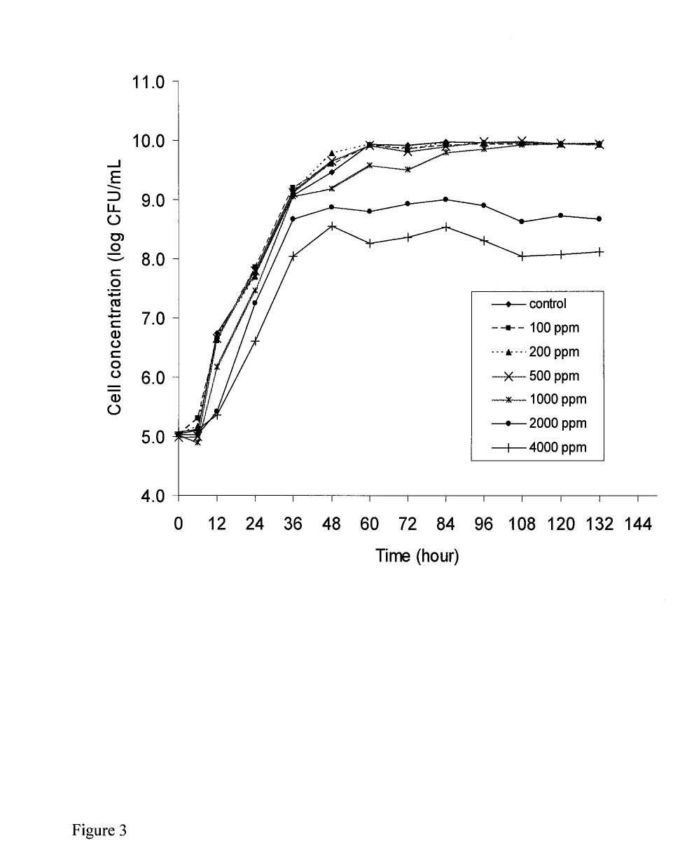

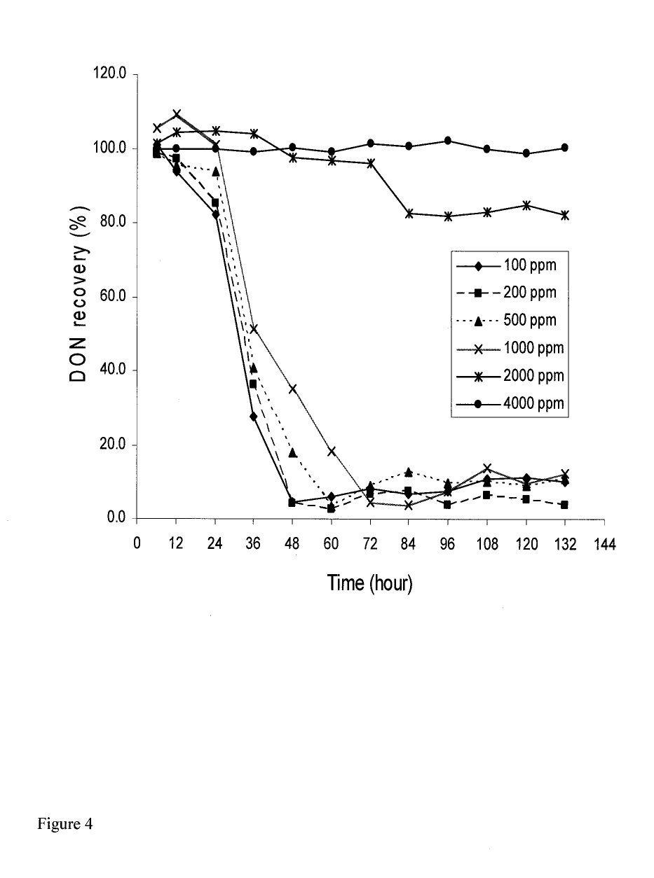

There was no reduction of DON by bacterial strain 040408-1 detected after 72 h incubations in CMB medium under anaerobic condition at both 23 and 37° C. Growth rate and DON reduction experiments were also conducted in CMB medium under aerobic conditions at 28° C. Results indicated that bacterial strain 040408-1 was capable of growth in the presence of various concentrations of DON (FIG. 3). However, DON reduction activity was inhibited in the presence of very high concentrations of DON in CMB medium (FIG. 4).

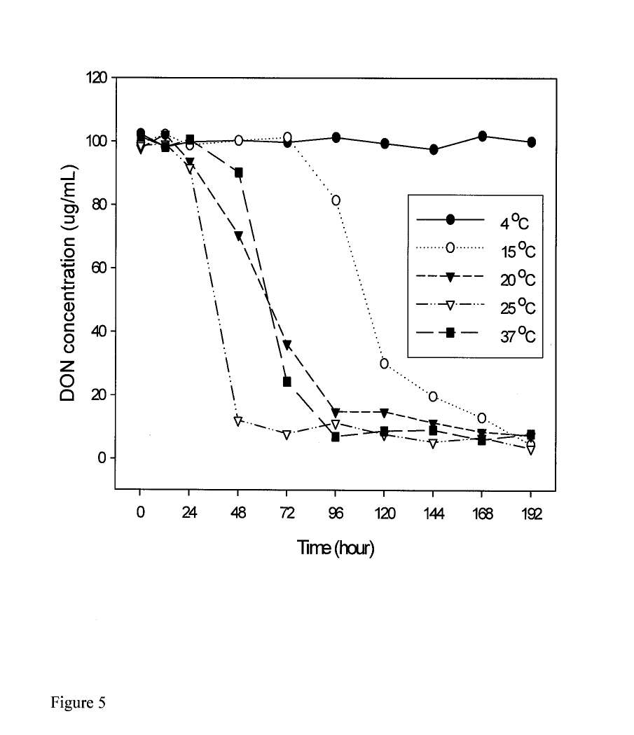

As will be understood by a person of skill in the art, temperatures also affect the growth and function of bacterial strain 040408-1 as shown in FIG. 5. The experimental results suggest that efficient incubation reaction conditions were about 28° C. However, temperatures between about 4° C. and about 37° C. were also shown to be capable of reducing DON. Accordingly, the present invention preferably contemplates the use of bacterial strain 040408-1 to reduce DON at a temperature of between about 15° C. and about 37° C., for example, but not limited to 15, 17, 19, 21, 23, 25, 27, 28, 29, 30, 32, 34, 36 and 37° C. or any temperature therein between.

However, in still further embodiments, the present invention contemplates the use of bacterial strain 040408-1 at temperatures higher than 37° C. or lower than 15° C.

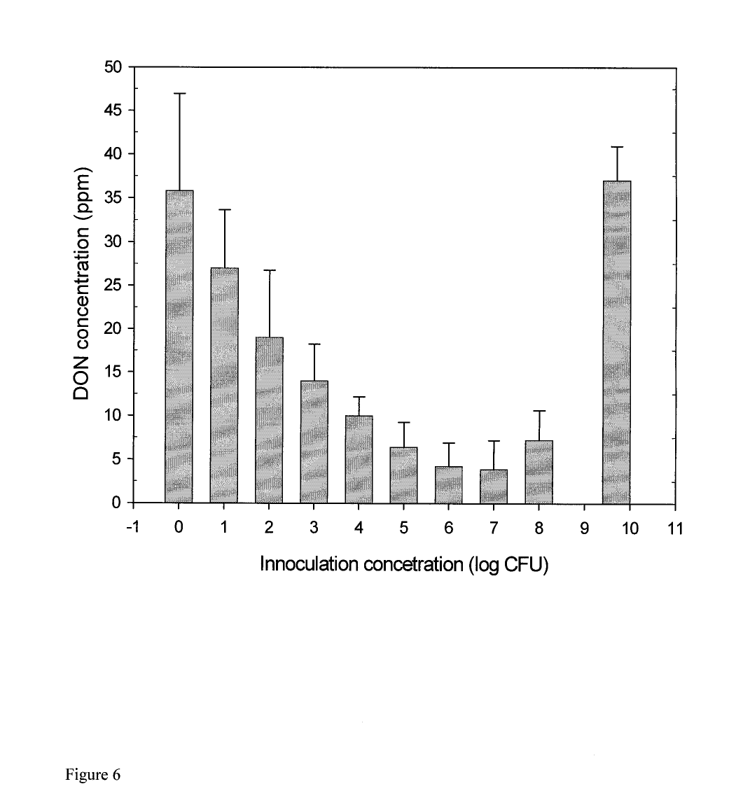

Effect of inoculation was determined using CMB cultures containing 100 μg/mL DON and bacterial strain 040408-1 with inoculation concentrations from 1×10⁰ up to 5×10⁹ CFU/mL at 28° C. with shaking at 200 rpm for 72 h. The coincidence of the growth of bacterial strain 040408-1 and the reduction of DON was also observed in each treatment (data not shown). At 72 h, DON concentrations were reduced to about 3.8-10.0 μg/mL (PLSD_{(0.05})=9.4 μg/mL) in treatments with concentrations of inoculation ranging from 1×10⁴ to 1×10⁸ CFU/mL (FIG. 6). DON concentrations of treatments with inoculation concentrations of 1×10⁰, 1×10¹, 1×10² and 1×10³ CFU/mL were reduced to below 10 μg/mL with continuous incubation (data not shown).

MM, MMY, MM-Purdue, Yeast+Glucose, BYE are media that are frequently used in the research of bacterial enzymes (Shima et al., 1997; Young et al., 2007). CMB, CMBPD were found to be suitable for screening DON-reducing microorganisms. CMB/WO/Salt, rice medium, malt extract are media that have similar nutrients to CMB. Nutrient broth, TSB, Lauria Bertani and MacConkey are common media for bacteria. Therefore, these media were then chosen for testing the growth and the function of bacterial strain 040408-1 in a culture condition of: 100 μg/mL DON, at 28° C., with shaking at 200 rpm for 72 h. Media CMB and Yeast+glucose gave lowest residue concentrations of DON in all these media, which were 4.5 and 0.0 μg/mL, respectively. 3-epi-DON was the major and consequent product of the reaction. Peak 5.0 and 3-keto-DON were found in certain media and their concentrations were low (about 5 μg/mL (Table 3).

Transformation Products of DON by Enriched Soils and Bacterial Strain 040408-1

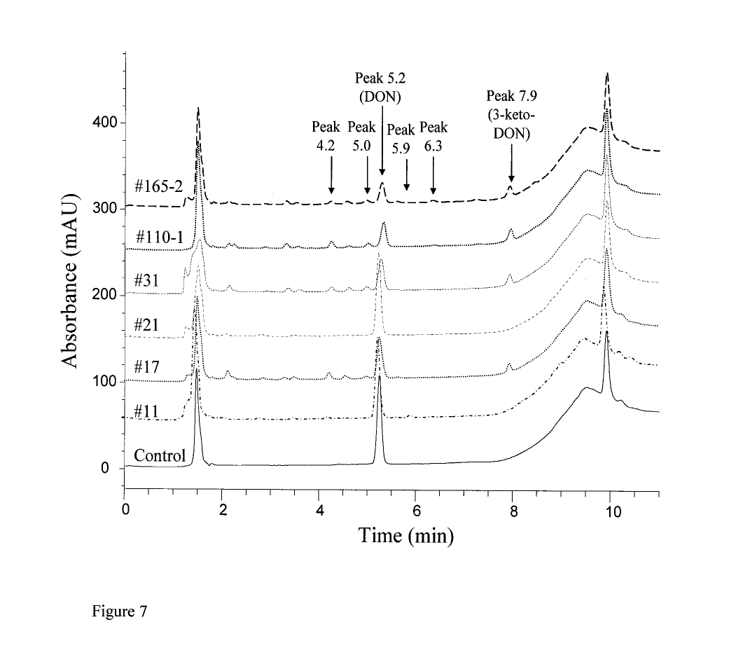

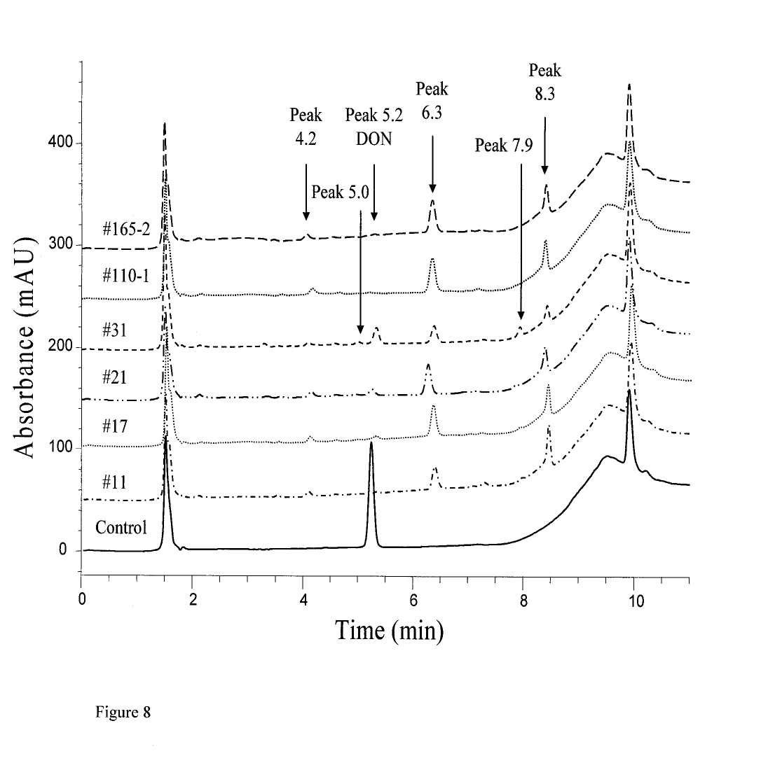

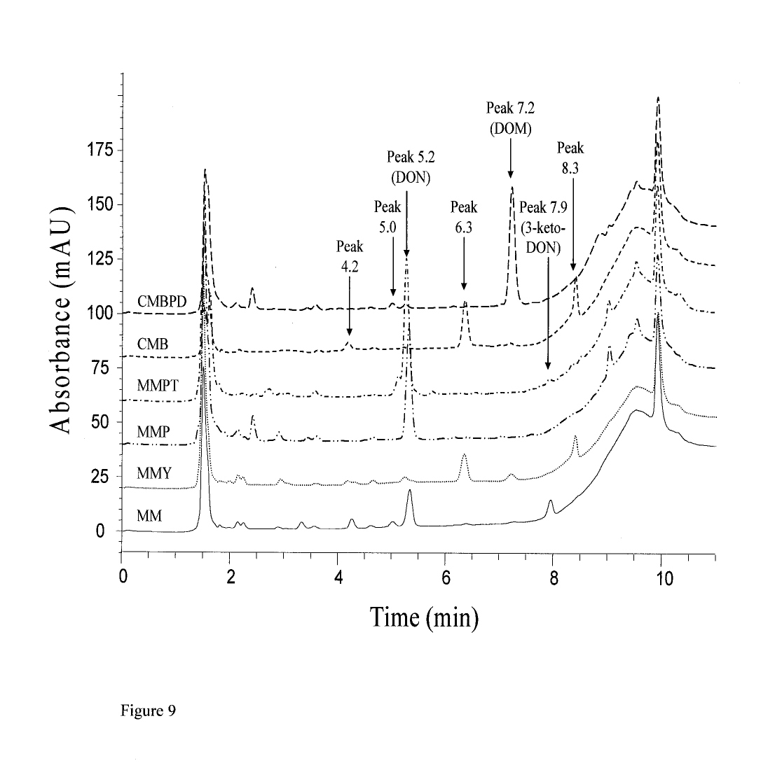

Soils in different media showed different DON reduction activities and gave different profiles of DON transformation products. A total of seven different transformation products of DON were found. They were named as Peaks 4.2, 5.0, 5.2, 5.9, 6.3, 7.2, 7.9, and 8.3, corresponding to their retention times in minutes. Different soils gave similar profiles of DON transformation products in the same medium. For example, in MM medium, the activities of DON transformation were generally low and the DON recoveries ranged from 24.5-93.7% under the conditions tested. The major transformation products were observed as peaks at the following retention times: 4.2, 5.0, and 7.9 min (FIG. 7). In CMB medium, the DON transformation activities were much higher than those in MM medium and the DON recoveries were between about 0-17.6%. The products showed peaks at 4.2, 6.3 and 8.3 min (FIG. 8). The profile of DON transformation in CMBPD medium was similar to that in CMB medium. However, a same soil was capable of transforming DON into different products in different media. FIG. 9 shows the HPLC chromatographs of transformation products of DON by the soil #165-2 in six different media.

The major product of DON transformation by bacterial strain 040408-1 was purified from the DON transformation culture using high speed countercurrent chromatography and identified as 3-epi-DON using NMR. Peak 5.9 has the same MW as DOM-1. The identities of the products eluting at 7.2 and 7.9 min were confirmed as DOM-1 and 3-keto-DON, respectively, by matching the retention time and UV and MS spectral data (Shima et al., 1997; Young et al., 2007).

Cytotoxicity of the Two Major Transformation Products of DON

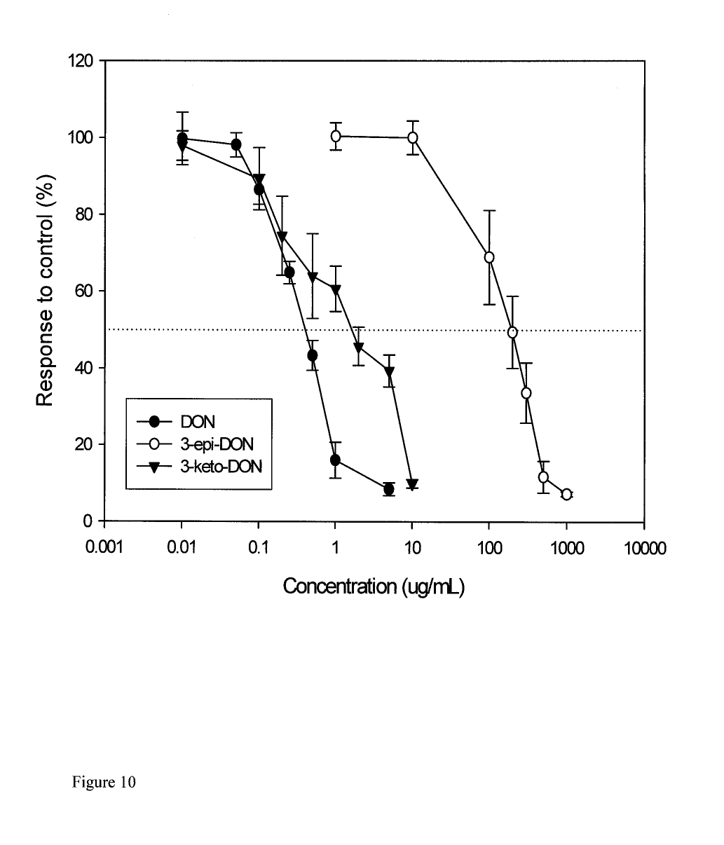

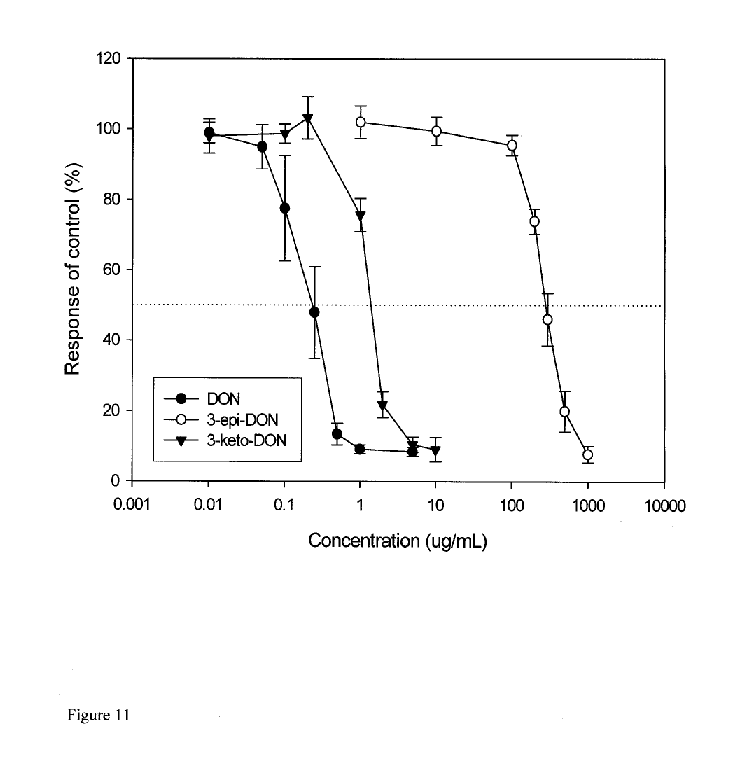

The cytotoxicity of DON, 3-epi-DON and 3-keto-DON was measured by MTT and BrdU bioassays in a concentration range from 0.0100-5.00 μg/mL (0.0338-16.9 mmol/L), 1.00-1000 μg/mL (3.38-3378 mmol/L) and 0.0100-10.0 μg/mL (0.0340-34.0 mmol/L). All tested compounds had a clear response to concentration in these two assays (FIGS. 10 and 11). The values of IC₅₀ and their relative values to DON were presented in Table 4. The IC₅₀ values of 3-epi-DON and 3-keto-DON were 357 and 3.03 times higher than that of DON on the base of the MTT bioassay and were 1181 and 4.54 times higher than that of DON on the basis of the BrdU bioassay.

The Capability of Bacterial Strain 040408-1 to Transform Other Trichothecenes

Bacterial strain 040408-1 was able to transform 3-acetyl-DON (MW=338, t_{R}=7.9 min) into four products with molecular weights (MW) of 296 (t_{R}=3.0 min), 308 (t_{R}=3.7 min), 308 (t_{R}=3.9 min) and 308 (t_{R}=6.4 min), 15-acetyl-DON (MW=338, t_{R}=7.8 min) into two products with MW of 338 (t_{R}=6.7 min) and 368 (t_{R}=8.1 min), Roridin A (MW=532, t_{R}=16.4) into two products with MW of 530, t_{R}=14.7 and 502, t_{R}=16.0).

### Example 2

Toxicity of Transformation Products of DON in an Animal Model

Female B6C3FI mice were obtained from Charles River Canada Inc (Montreal, Canada). Mice were housed in pairs in plastic cages under conditions meeting the requirements of the Canadian Council for Animal Care and were acclimatized for one week before the start of the study. 2014 Teklad Global 14% Protein Rodent Maintenance Diet (Harlan Laboratories, Inc., Quebec, Canada) and water were provided ad libitum before and throughout the study.

The experiment included 10 mice per group for each of the following treatments: Control (solvent control, free of toxin); 2 mg/kg DON; 25 mg/kg 3-epi-DON, and 100 mg/kg 3-epi-DON. There were no significant differences in starting body weights for any of the groups studied (P>0.05). Each mouse received a single daily gavage dose with a 20-gauge stainless-steel gavage needles (Popper and Sons, Inc., New Hyde Park, N.Y., USA) for 14 consecutive days. Body weights were monitored daily throughout the study. The food consumption was measured every 3 or 4 days. On the final day of the study, all mice were anaesthetized with isoflurane (Aerrane®, Anaquest, Ontario, Canada) and exsanguinated by cardiac puncture. Organ weights were recorded for heart, liver, kidneys, spleen, and thymus.

Toxicological Effects on Mouse Organs of 3-epi-DON

All mice were healthy in appearance and demeanour for the duration of the study. On the day of necropsy, final body weights of mice in the treatment of 2 mg/kg DON appeared lower than those of Control, 25 mg/kg 3-epi-DON, and 100 mg/kg 3-epi-DON treatments (P=0.095) (Table 5). All tested organ weights were expressed relative to body weights. Heart weights were not significantly different among the four treatments at the time of necropsy (P=0.111). Spleen weights of mice in the treatment of 2 mg/kg DON appeared lower than those of other three treatments (P=0.094). Weights of liver (P <0.001), kidney (P<0.001) and thymus (P<0.001) of mice in 2 mg/kg DON treatment were significantly lower than those of Control. However, there were no significant difference among the Control and the two 3-epi-DON treatments at 25 mg/kg and 100 mg/kg, respectively, in weights of liver, kidney, spleen and thymus (Table 5).

### Example 3

Calibration Curve and Preparation of Inoculum

An initial calibration curve of 040408-1 was made using a dilution plating technique and turbidity measurements. Bacterial isolate 040408-1 from pure culture was grown on 1 ml of CMB on a rotary shaker at 28° C. for 24 h with shaking at 200 rpm. From this original suspension, serial two-fold dilutions were made and optical density (OD) readings performed at 620 nm for each resulting suspension using a Ultrospec 3100 Pro UV/Visible spectrophotometer (Biochrom Ltd., Cambridge, UK) until OD was approximately 0.10. Additionally, each new suspension was used to make a 10-fold dilution series up to 10⁻³, from which 100 μL of supernatant was inoculated and spread onto corn meal agar plates. The plates were incubated in the dark at 28° C. for 3 days and the number of forming colonies units per millilitre (CFU mL⁻¹) was determined by plate counting and the number of CFU for each two fold-dilution was extrapolated. The calibration curve was then plotted using the number of CFU mL⁻¹ vs the OD readings.

Bacterial isolate 040408-1 from original plates was incubated for 24 h at 28° C. in CMB and diluted in autoclaved water to ca 10⁶ CFU mL⁻¹, was used as the inoculum. All test microbial cultures were spiked with DON solution dissolved in water to a final concentration of 50 mg L⁻¹.

Chemicals

Deoxynivalenol standard was purchased from Sigma (St Louis, Mo.); all solvents were LC-grade (Caledon Labs Ltd, Georgetown, ON, Canada). Mineral and media ingredients were purchased from Fisher Scientific (Fair Lawn, N.J., USA), Sigma Chemical Co. (St. Louis, Mo., USA), Becton, Dickinson and Company (Le Pont de Claix-Cedex, France) or Fluka Chemie (Buchs, Switzerland).

Assessment of Culture Conditions

Culture conditions including incubation temperature and medium pH were examined to determine their effects on bacterial growth and DON-biotransforming activity. The effect of incubation temperature was examined by incubating bacteria in CMB at 5, 10, 15, 20, 25, 30, 35 and 40° C. for 48 h under aerobic conditions. Aliquots of CMB were adjusted to pH 3.0, 4.0, 5.0, 6.0, 7.0, 8.0, 9.0 and 10.0 with NaOH or HC1 1N before sterilization to examine the effect of starting pH. After inoculation with the test organism and under aerobic conditions, microbial cultures were incubated at 28° C. with shaking (200 rpm) for 48 h. Growth and DON biotransformation were then determined. CMB was prepared according to previous experiments (Example 1). Corn meal (40 g) was soaked for approximately 4 h in 1 l of deionized water. Before filtering, minerals were added including (NH₄)₂SO₄, 3 g; K₂HPO₄, 1.0 g; MgSO₄, 0.5 g; K₂SO₄, 0.5 g; FeSO₄, 0.1 g; MnSO₄, 0.07 g and yeast extract, 5 g.

Assessment of Culture Media

To examine the effect of media components on bacterial growth and DON-biotransforming activity, different carbon and nitrogen sources, with and without the addition of minerals were evaluated. The carbon sources tested were glucose (GLU) (a monosaccharide), sucrose (SUC) (a disaccharide), and corn starch (STA) (a polysaccharide). The nitrogen sources used were of two types: organic sources, which included corn steep liquor (CSL), peptone (PEP), yeast extract (YEA), and urea (URE); and the inorganic sources ammonium sulphate (SUL) and ammonium nitrate (NIT). The concentration of the carbon and nitrogen sources was 10 g L⁻¹. The minerals used and their concentration per liter of distilled water were the same as above. As shown in Table 6, 18 different media were evaluated and CMB was used as a reference control. All media were homogenized and autoclaved at 121° C. for 15 min. After inoculation with the test organism, microbial solutions were incubated under aerobic conditions at 28° C. with shaking (200 rpm) for 72 h. Growth and DON biotransformation in microbial cultures were then determined.

Preparation of Samples for Bacterial Growth and DON Biotransformation Determination

After incubation, optical density (OD) readings were performed at 620 nm using a Ultrospec 3100 Pro UV/Visible spectrophotometer (Biochrom Ltd., Cambridge, UK) on each sample to establish the bacterial growth using a calibration curve previously established. The solution was mixed with methanol at 2:1 ratio using a vortexer for 1 minute and let stand for 2 minutes. The final solution was centrifuged at 4000 rpm for 5 minutes, filtrated using a 0.45 mm pore size membrane filter immediately before DON analysis by LC/MS.

Analysis of DON and Metabolites by LC/MS

For DON analysis 20 gL of sample was injected onto a 4.6×150 mm LC column packed with Agilent Zorbax Eclipse XDB-C18, 3.5 μm particle size. The column was eluted at 1 mL/min with a gradient of methanol—water changing from 25% methanol to 41% over 5 min, held at 41% for 3 min, then back to 25% over 1 min and held at 25% for 3 min. Starting materials and products were detected with a Finnigan Spectra System UV6000LP ultraviolet (UV) detector and a Finnigan LCQ Deca ion trap MS operated in the positive ion atmospheric pressure chemical ionization (APCI) mode. The mass spectrometer was tuned for maximum response for DON. Machine operating conditions were as follows: shear gas and auxiliary flow rates were set at 80 and 0 (arbitrary units); voltages on the capillary, tube lens offset, multipole 1 offset, multipole 2 offset, lens, and entrance lens were set at 15.00, 30.00, −5.00, −7.00, −16.00, and −60.00 V, respectively; capillary and vaporizer temperatures were set at 200° C. and 450° C., respectively; and the discharge needle current was set at 10 μA. Identities of compounds were confirmed by the congruence of retention times and UV and MS spectral data with those of authentic standards. DON, 3-epi-DON and 3-keto-DON were quantified on the basis of integrated peak areas using MS selected ion monitoring (SIM) at m/z 231, 249, 267, 279, and 297 for DON and 3-epi-DON and m/z 247, 261, 277, and 295 for 3-keto-DON. It was assumed that the molar response factor for each metabolite was equal to that of DON. The percentage of DON biotransformation was estimated by subtracting the remaining DON after incubation from the initial concentration, multiplied×100.

Rate of DON Biotransformation

Bacterial isolate 040408-1 suspended in CMB (106 CFU mL⁻¹) was spiked with 50 mg L⁻¹ DON and incubated at 28° C. under aerobic conditions at 200 rpm. Samples of the microbial culture were taken every 12 hours during a period of 48 h to determine changes in concentrations of DON and metabolites produced.

Statistical Analysis

Each experiment was conducted in duplicate with at least a triplicate determination for each sample. The experimental design was completely randomized and the effect of the different media was determined by one-way ANOVA using Statistix® for Windows version 2.0. Variable means from treatments showing significant differences in the ANOVA were compared using the Tukey's test (P<0.05).

Effect of Temperature

Table 7 shows the growth and DON biotransformation by bacterial isolate 040408-1 at various culture temperatures in CMB. The highest 040408-1 growth (P<0.05) was observed in CMB at temperatures of 30 and 35° C. followed by 25 and 20° C. These four groups also showed good DON biotransformation rates (P<0.05). As the cultivation temperature decreased from 20 to 5° C., growth of the test organism was slower and little DON biotransformation was observed, whereas at 40° C., even though the production of biomass was significant (P=0.05), little DON biotransforming activity was detected.

Effect of pH

As shown in Table 8, among the various pH's tested, growth of bacterial isolate 040408-1 was substantial (over 1.0×10⁹ CFU mL⁻¹) in media with an initial pH between 6.0 and 7.1 (P<0.05). The bacteria showed little or no appreciable growth in media with an initial pH of 3.0, 4.2 or 5.2, while at pH of 8.4, 9.3 and 10.2, although the growth increased, it was not significantly different (P<0.05) from that observed at lower pH's. Adjusting the initial pH of the culture broth to 7.1 resulted in substantial DON biotransforming activity by 040408-1 (95.1%) followed by the activity at pH 6.0 and 8.4 (77.5% and 37.8 respectively). Little or no DON biotransformation was detected in media having an initial pH of 3.0, 4.2, 5.2, 9.3 and 10.2 (P<0.05).

Effect of Nutrients in Culture Media

Table 9 shows the growth and the percentage of DON biotransformation of the bacterial isolate 72 h after its culture in media containing various carbon and nitrogen sources with and without minerals added. When no minerals were added to test media, the highest growth of 040408-1 was obtained with YEA (5.3×10⁹ CFU mL⁻¹) followed by CSL and PEP. No differences were found between GLU, SUC, STA, URE, SUL or NIT (P<0.05), and the final concentration was lower than 1.6×10⁷ CFU mL⁻¹ in all cases. The highest DON biotransforming activity (87.7%) was detected when YEA was used as media while almost no DON biotransformation was detected when GLU, SUC, STA, URE, SUL and NIT were used with less than 1.5% in all cases. DON biotransforming activity was also observed with PEP and CSL with values of 49.3% and 17.1%, respectively. As expected, CMB showed significantly higher values on both growth and DON biotransformation (7.7×10⁹ CFU ml⁻¹ and 97.8 respectively).

When minerals were added to media, YEA yielded the highest growth of bacterial isolate 040408-1 (8.7×10⁹ CFU mL⁻¹), followed by CSL and PEP. STA, URE, SUL and NIT showed the lowest growth rates with values below 9.9×10⁶ CFU mL⁻¹, while GLU and SUC showed higher values with 1.2 and 4.0×10⁷ CFU mL⁻¹, respectively. In regards to DON biotransformation, CSL, PEP and YEA exhibited higher percentages (99.5%, 99.2% and 84.4% respectively) compared to the values obtained when GLU, SUC, STA, URE, SUL and NIT were used as test media (P<0.05).

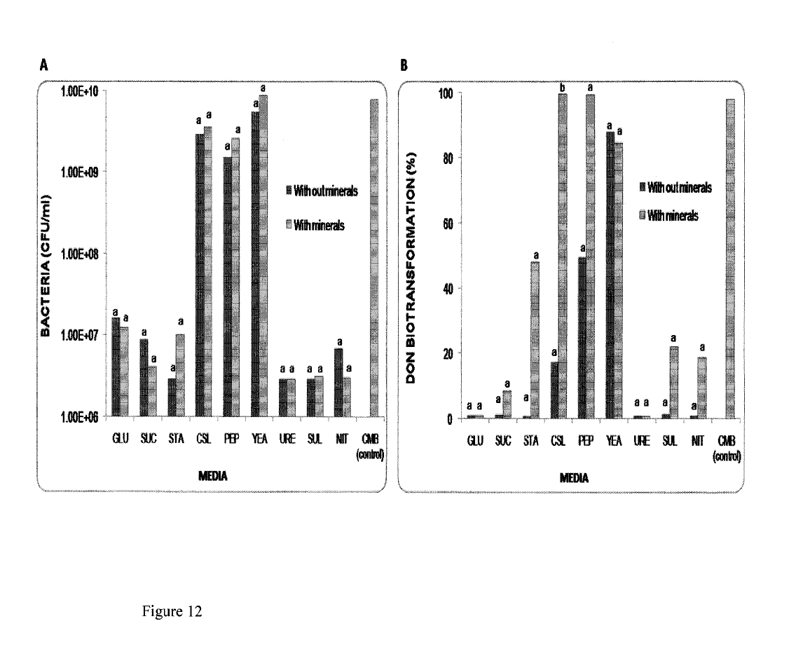

FIG. 12A and 12B compare growth and DON biotransformation between media with and without minerals. Addition of minerals had no significant effect on bacterial growth, while in DON biotransformation only CSL showed a significant (P<0.05) improvement with the addition of minerals (from 17.1 to 99.5%).

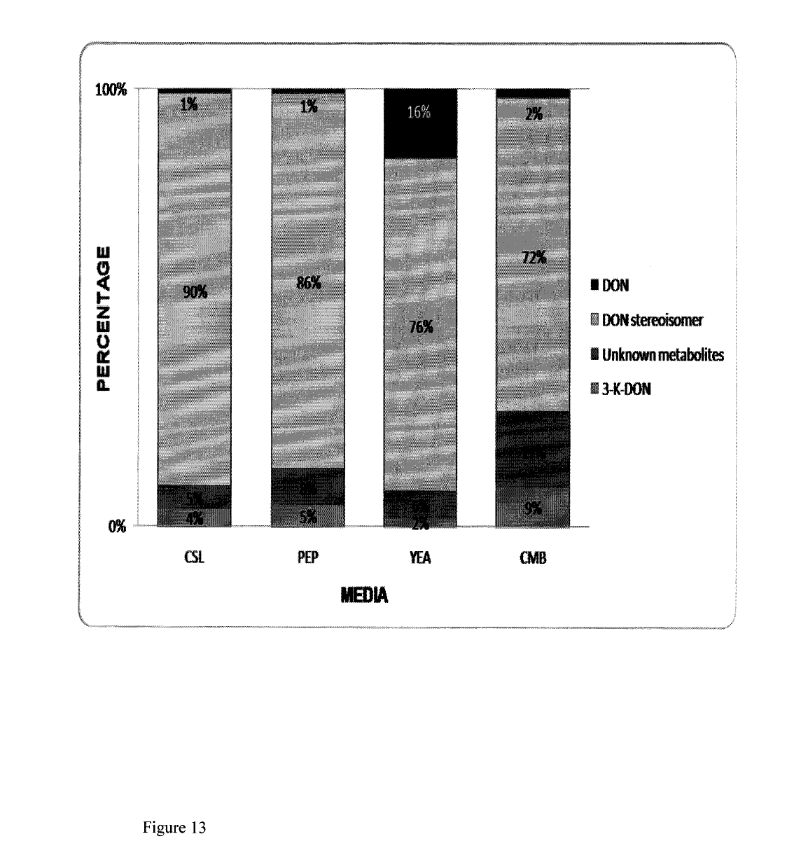

Profiles reflecting the amount (in percentage) of reaction products obtained from DON biotransformation for media CSL, PEP, YEA supplemented with minerals, and CMB are shown in FIG. 13. After 72 h of incubation, DON was converted (99%) to metabolites (except in YEA media). As expected the two already known major metabolites 3-epi-DON (DON steroisomer) and 3-keto-DON were identified in all samples. The main product of the biotransformation was 3-epi-DON with more than 72% in all cases while the 3-keto-DON metabolite amounted to 10% or less. An unknown compound was also detected, especially in CMB medium where the percentage was as high as 17%. Fragments observed in the mass spectrum of the compound included m/z 247, 277, 291 and 309 (molecular weight of the compound is 308 Da). Although differences in the profiles were observed on DON, 3-keto-DON and unknown compounds, no significant differences were found in the percentages of 3-epi-DON at p=0.05 level.

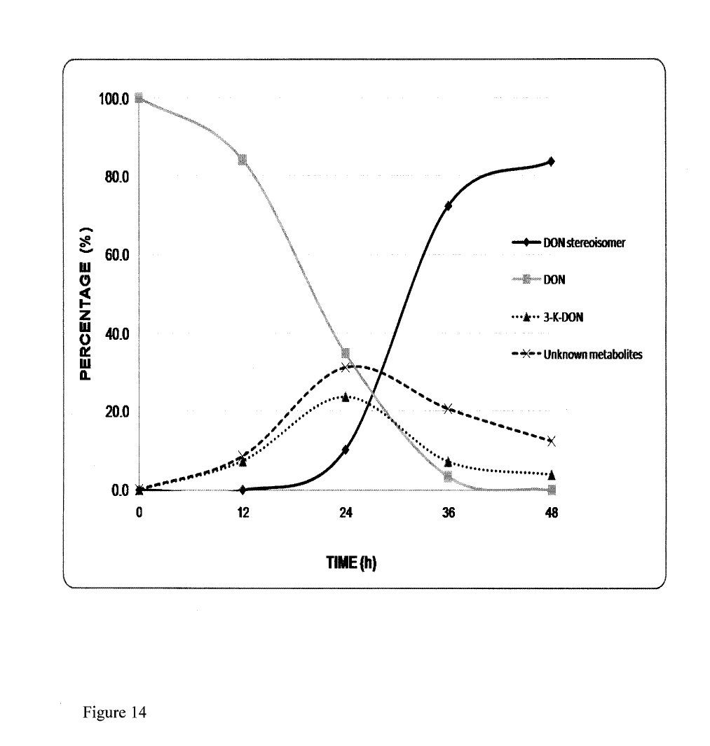

FIG. 14 shows the changes in levels of DON and biotransformation products over an incubation time of 48 h at 28° C. in CMB with minerals added. As observed in the DON biotransformation curve, the level of 3-epi-DON progressively increased linearly with time and DON was biotransformed to products after 36 hours hr. The unknown metabolites as well as 3-keto-DON reached maximum levels at 24 h under the conditions tested but tended to diminish by 48 h.

The present invention has been described with regard to one or more embodiments. However, it will be apparent to persons skilled in the art that a number of variations and modifications can be made without departing from the scope of the invention as defined in the claims.

References (All citations are Herein Incorporated by Reference):
Bata, A., and R. Laszitity. 1999. Detoxification of mycotoxin-contaminated food and feed by microorganisms. Trends Food Sci. Technol. 10:223-228.
Bhat R V, Beedu S R, Ramakrishna Y, Munshi K L. 1989. Outbreak of trichothecene mycotoxicosis associated with consumption of mould-damaged wheat in Kashmir Valley, India. Lancet 7:35-37.
Bridson, E. Y. and Bracker, a. 1970. Design and formulation of microbial culture media. Methods in Microbiology, vol 3A, pp 229-295.
Canady, R., Coker, R., Egan, S. K., Krska, R., Kuiper-Goodman, T., and Olsen, M. (2001). Deoxynivalenol (DON), Safety evaluation of certain mycotoxins in food. Joint FAO/WHO Expert Committee on Food Additives (JECFA), FAO Food and Nutrition Paper 74, 419-555.
Cardwell, K. F., Desjardins, A., Henry, S. H., Munkvoild, G., and Robens, J. (2001). Mycotoxins: The Cost of Achieving Food Security and Food Quality. http://www.apsnet.org/online/feature/mycotoxin Accessed on Sep. 1, 2008.
Carrow, R. N., Waddington D. V., and Rieke, P. E. 2001. Turfgrass soil fertility and chemical problems: Assessment and management. Ann Arbor Press, Chelsea, MI, pp274.
CAST—Council for Agricultural Science and Technology-, 2003. Mycotoxins: Risks in Plant, Animal, and Human Systems. Task Force Report No. 139, Ames, Iowa.
CAST (Council for Agricultural Science and Technology) (1989). Mycotoxin: Economic and Health Risks. Task Force Report No. 116.
Cetin, Y., and Bullerman, L. B. (2005). Cytotoxicity of Fusarium mycotoxins to mammalian cell cultures as determined by the MTT bioassay. Food and Chemical Toxicology 43, 755-764.
Charmley, E., Trenholm, H. L., Thompson, B. K., 1993. Influence of level of deoxynivalenol in the diet of dairy cows on feed intake, milk production and its composition. J. Dairy Sci. 76, 3580-3587.
Commission Regulation (EC) (2001). Commission Regulation (EC) No 466/2001 of 8 Mar. 2001 setting maximum levels for certain contaminants in foodstuffs, Brussel.
Conková, E., Laciaková, A., Kovac, G., and Seidel, H. (2003). Fusarial Toxins and their Role in Animal Diseases. The Veterinary Journal 165, 214-220.
Conrady-Lorck, S., Gareis, M., Feng, X. C., Amselgruber, W., Forth, W. and Fichtl, B., 1988. Metabolism of T-2 toxin in vascularly autoperfused jejunal loops of rats. In: Toxicology and Applied Pharmacology 94: 23-33.
Cousin, M. A., Riley, R. T., Pestka, J. J., 2005. Foodborne mycotoxins: chemistry, biology, ecology, and toxicology. In: Fratamico, P. M., Bhunia, A. K. (Eds.), Foodborne Pathogens: Microbiology and Molecular Biology. Horizon Scientific Press, Ltd., Norfolk, UK, pp. 163-226.
Crueger, W. and Crueger A. 1990. Biotechnology - A textbook of industrial microbiology. Second edition. Thomas D. Brock (Editor of the English edition). Sinauer Associates, Inc. Sunderland, Mass. 01375.357 Pp.
D'Mello, J. P. F., Placinta, C. M., and Macdonald, A. M. C. (1999). Fusarium mycotoxins: A review of global implications for animal health, welfare and productivity. Animal Feed Science and Technology 80, 183-205.
Dänicke, S., Valenta, H., Gareis, M., Lucht, H. W., and von Reichenbach, H. (2005). On the effects of a hydrothermal treatment of deoxynivalenol (DON)-contaminated wheat in the presence of sodium metabisulphite (Na2S2O5) on DON reduction and on piglet performance. Animal Feed Science and Technology 118, 93-108.
Desjardins, A. E. (2006). Fusarium Mycotoxins: Chemistry, Genetics, and Biology. American Phytopathological Society, St. Paul.
DeVries, J. W., Trucksess, M. W., Jackson, L. S., 2002. Mycotoxins and Food Safety: Proceedings of an American Chemical Society Symposium held in Washington, D.C., USA, on 21-23 August 2000. Kluwer Academic Publishers, Dordrecht, Netherlands, p. 295.
Diaz, D. E., Hagler Jr, W. M., Hopkins, B. A., and Whitlow, L. W. (2002). Aflatoxin binders I: in vitro binding assay for aflatoxin B1 by several potential sequestering agents. Mycopathologia 156, 223-226.
Diaz, G. J. (2002) Evaluation of the efficacy of a feed additive to ameliorate the toxic effects of 4, 15- diacetoxiscirpenol in growing chicks. Poultry Sci., 81, 1492-1495.
Drew, S., and Wallis, D. 1983. Regulation of secondary metabolism and keys to its manipulation. In: Secondary metabolism and differentiation in fungi. Bennett J and Ciegler A (eds). Marcel Dekker Inc. New York. N.Y.
Ehling G, Cockburn A, Snowdon P, Buchhaus H, 1997. The significance of the Fusarium toxin deoxynivalenon (DON) for human and animal health. Cereal Research Commun 25: 433-447.
Epstein, W. (2003). The roles and regulation of potassium in bacteria. Prog. Nucleic Acid Res. Mol. Biol. 75: 293-320.
Eriksen G S, Alexander J (eds.), 1998. Fusarium toxins in cereals—a risk assessment. Nordic Council of Ministers; Temallord 1998: 502, pp. 7-27 and 45-58; Copenhagen.
Eriksen, G. S., and Pettersson, H. (2004). Toxicological evaluation of trichothecenes in animal feed. Animal Feed Science and Technology 114, 205-239.
Eriksen, G. S., Pettersson, H., and Lundh, T. (2004). Comparative cytotoxicity of deoxynivalenol, nivalenol, their acetylated derivatives and de-epoxy metabolites. Food and Chemical Toxicology 42, 619-624.
Feng, Y. 2008. Soil microbiology. In: Encyclopedia of Soil Science. Chesworth, W (ed). Encyclopedia of Earth Science Series, Springer. Dordrecht, The Netherlands, pp 676.
Forsyth, D. M., Yoshizawa, T., Morooka, N., and Tuite, J. 1977. Emetic and refusal activity of deoxynivalenol to swine. Appl. Environ. Microbiol. 34:547-552.
Fraleigh, S., Bungay H., and Fiechter, A. 1989. Regulation of oxidoreductive yeast metabolism by extracellular factors. Review. Journal of Biotechnology, 12: 185-198
Fuchs, E., Binder, E. M., Heidler, D. and Krska, R., 2002. Structural characterization of metabolites after the microbial degradation of type A trichothecenes by the bacterial strain BBSH 797. Food Additives and Contaminants 19: 379-386.
Galvano, F., Pietri, A., Bertuzzi, T., Piva, A., Chies, L., and Galvano, M. (1998). Activated carbons: in vitro affinity for ochratoxin A and deoxynivalenol and relation of adsorption ability to physicochemical parameters. Journal of Food Protection 61, 469-475.
Guan, S., He, J., Young, J. C., Zhu, H., Li, X., Ji, C., & Zhou, T. (2009). Transformation of trichothecene mycotoxins by microorganisms from fish digesta. Aquaculture, 290, 290e295.
He, J., Yang, R., Zhou, T., Tsao, R., Young, J. C., Zhu, H., Li, X.-Z., and Boland, G. J. (2007). Purification of deoxynivalenol from Fusarium graminearum rice culture and mouldy corn by high-speed counter-current chromatography. Journal of Chromatography A 1151, 187-192.
He, P., Young, L.G. and Forsberg, C., 1992. Microbial transformation of deoxynivalenol (vomitoxin). Applied and Environmental Microbiology 58: 3857-3863.
Heidler, D and Schatzmayr G. 2003. A new approach to managing mycotoxins. World Poultry—Reed Volume 19. Available at: www.AgriWorld.nl
Horvath, I. and Varga, M., 1961. Enzymatic Inactivation of trichothecin and crotocin. Nature 192: 88.
Hughes D. M., Gahl M. J., Graham C. H. and Grieb S. L., 1999. Overt signs of toxicity to dogs and cats of dietary deoxynivalenol. Journal of Animal Science, Vol 77, Issue 3 693-700
Huwig, A., Freimund, S., Käppeli, O., & Dutler, H. (2001). Mycotoxin detoxication of animal feed by different adsorbents. Toxicology Letters, 122, 179-188.
JECFA56th. 2001. Safety Evaluation of Certain Mycotoxins in Food. WHO Food additives Series, No. 47, Geneva, Switzerland.
Jelinek, C. F., Pohland, A. E., and Wood, G. E. 1989. Review of mycotoxin contamination. Worldwide occurrence of mycotoxins in foods and feeds. An update. J. Assoc. Off. Anal. Chem. 72:223-230.
Jouany, J. P. 2007. Methods for preventing, decontaminating and minimizing the toxicity of mycotoxins in feeds./Animal Feed Science and Technology 137: 342-362
Karlovsky, P. (1999). Biological Detoxification of Fungal Toxins and its Use in Plant Breeding, Feed, and Food Production. Natural Toxins 7, 1-23.
Kendra, D. F., and Dyer, R. B. (2007). Opportunities for biotechnology and policy regarding mycotoxin issues in international trade. International Journal of Food Microbiology 119, 147-151.
Kiessling, K.-H., H. Pettersson, K. Sandholm, and M. Olsen. 1984. Metabolism of aflatoxin, ochratoxin, zearalenone, and three trichothecenes by intact rumen fluid, rumen protozoa, and rumen bacteria. Appl. Environ. Microbiol. 47:1070-1073. Matsushima, T., Okamoto, E., Miyagawa, E., Matsui, Y., Shimizu, H. and Asano, K., 1996. Deacetyllation of diacetoxyscirpenol to 15-acetoxyscirpenol by rumen bacteria. Journal of General and Applied Microbiology 42: 225-234.
King, R. R., R. E. McQueen, D. Levesque, and R. Greehalgh. 1984. Transformation of deoxynivalenol (vomitoxin) by rumen microorganisms. J. Agric. Food Chem. 32:1181-1183.
Kouadio, J. H., Mobio, T. A., Baudrimont, I., Moukha, S., Dano, S. D., and Creppy, E. E. (2005). Comparative study of cytotoxicity and oxidative stress induced by deoxynivalenol, zearalenone or fumonisin B1 in human intestinal cell line Caco-2. Toxicology 213, 56-65.
Kuiper-Goodman T. 1994. Prevention of human mycotoxicosis through risk assessment and risk management. In: Miller J D, Trenholm H L, editors. Mycotoxins in grain. Mycotoxins in grain. St. Paul, Minn.: Eagan Press. pp 439-470.
Kushiro, M. 2008. Effects of Milling and Cooking Processes on the Deoxynivalenol Content in Wheat. International Journal of Molecular Sciences. Int. J. Mol. Sci Vol. 9, 2127-2145.
Leeson, S., Diaz, G., Summers, J. D., 1995. Poultry Metabolic Disorders and Mycotoxins. University Books, Guelph, Canada, pp. 191-192.
Lemke, S. L., Ottinger, S. E., Mayura, K., Ake, C. L., Pimpukdee, K., Wang, N., and Phillips, T. D. (2001). Development of a multi-tierid approach to the in vitro prescreening of clay-based enterosorbents. Animal Feed Science and Technology 93, 17-29.
Miller J. D., ApSimon J. W., Blackwell B. A., Greenhalgh R., and Taylor A. 2001. Deoxynivalenol: a 25 year perspective on a trichothecene of agricultural importance, p. 310-320. In B. A. Summerell, J. F. Leslie, D. Backhouse, W. L. Bryden, and L. W. Burgess (ed.), Fusarium, Paul E. Nelson Memorial Symposium. APS Press, St. Paul, Minn.
Miller, J. David (2008). Mycotoxins in small grains and maize: Old problems, new challenges', Food Additives & Contaminants: Part A,25:2,219-230
Morooka N, Uratsuji N, Yoshizawa T, Yamamoto H. 1972. Studies on the toxic substances in barley infected with Fusarium species. Journal of Food Hygiene Society Japan 13:368-375.
Nelson, P. E. (2002). Fusarium-Paul E. Nelson Memorial Symposium. The American Phytopathological Society, St. Paul.
Palumbo, Jeffrey D., O'Keeffe, Teresa L. and Abbas, Hamed K. (2008). Microbial interactions with mycotoxigenic fungi and mycotoxins, Toxin Reviews. 27:3,261-285
Peppler, H. J. (1982) Yeast extracts. In: Fermented Foods by A. H. Rose (Editor). Academic Press Inc. New York, pp. 293-312.
Pestka, J., and Smolinski, A. (2005). Deoxynivalenol: Toxicology and potential effects on humans. Journal of Toxicology and Environmental Health Part B 8, 39-69.
Pestka, J. J. (2007). Deoxynivalenol: Toxicity, mechanisms and animal health risks. Animal Feed Science and Technology 137, 283-298.
Pittet, A. (1998). Natural occurrence on mycotoxins in foods and feeds-an updated review. In Revue de Medecine Veterinaire (J. le Bars, Galtier, P., Burgat, V., Guerre, P., Ed.), pp. 479-492.
Placinta, C. M., D'Mello, J. P. F., and Macdonald, A. M. C. (1999). A review of worldwide contamination of cereal grains and animal feed with Fusarium mycotoxins. Animal Feed Science and Technology 78, 21-37.
Pollmann, D. S., B. A. Koch, L. M. Seitz, H. E. Mohr, and G. A. Kennedy. 1985. Deoxynivalenol-contaminated wheat in swine diets. J. Anim. Sci. 60:239-247.
Prelusky, D. B., Rotter, B. A., Rotter, R. G., 1994. Toxicology of mycotoxins. In: Miller, J. D., Trenholm, H. L. (Eds.), Mycotoxins in Grain: Compounds Other than Aflatoxin. Eagan Press, St. Paul, Minn., pp. 359-403.
Ramirez, M. L., Chulze, S., and Magan, N. (2006). Temperature and water activity effects on growth and temporal deoxynivalenol production by two Argentinean strains of Fusarium graminearum on irradiated wheat grain. International Journal of Food Microbiology 106, 291-296.
Rotter, B. A., Prelusky, D. B., and Pestka, J. J. (1996). Toxicology of deoxynivalenol (vomitoxin). Journal of Toxicology and Environmental Health 48, 1-34.
Schaafsma, A. W. (2002). Economic changed imposed by mycotoxins in food grains: Case study of deoxynivalenol in winter wheat. In Mycotoxins and Food Safety. Advances in Experimental Medicine and Biology, No. 504 (J. W. DeVries, M. W. Trucksess, and L. S. Jackson, Eds.), pp. 271-276, New York, NY: Kluwer.
Scott, P. M. 1991. Possibilities of reduction or elimination of mycotoxins present in cereal grains. In Cereal grain. Mycotoxins, fungi and quality in drying and storage, ed. J. Chelkowski, pp. 529-572. Amsterdam: Elsevier.
Sergent, T. e. e., Parys, M., Garsou, S., Pussemier, L., Schneider, Y.-J., and Larondelle, Y. (2006). Deoxynivalenol transport across human intestinal Caco-2 cells and its effects on cellular metabolism at realistic intestinal concentrations Toxicology Letters 164, 167-176.
Shima, J., Takase, S., Takahashi, Y., Iwai, Y., Fujimoto, H., Yamazaki, M., and Ochi, K. (1997). Novel detoxification of the trichothecene mycotoxin deoxynivalenol by a soil bacterium isolated by enrichment culture. Applied and Environmental Microbiology 63, 3825-3830.
Smith, J. E.; Henderson, R. S. 1991. Mycotoxins and Animal Foods, 1st Ed.; CRC press: Boca Raton, Fla., USA. Pp. 875.
Statistix® for Windows. 1998. User's Manual. Analytical Software, Tallahasee, FL.
Swamy, H. V., Smith, T. K.,Macdonald, E. J., Boermans, H. J. and Squires, E. J. (2002) Effects of feeding a blend of grains naturally contaminated with Fusarium mycotoxins on swine performance, brain regional neurochemistry, and serum chemistry and the efficacy of a polymeric glucomannan mycotoxin adsorbent. J. Anim. Sci., 80, 3257-3267.
Swanson, S. P., Helaszek, C., Buck, W. B., Rood Jr., H. D. and Haschek, W. M., 1988. The role of intestinal microflora in the metabolism of trichothecene mycotoxins. Food and Chemical Toxicology 26: 823-829.
Swanson, S. P., Nicoletti, J., Rood Jr., H. D., Buck, W. B., Cote, L. M. and Yoshizawa, T., 1987a. Metabolism of three trichothecene mycotoxins, T-2 toxin, diacetoxyscirpenol and deoxynivalenol, by bovine rumen microorganisms. Journal of Chromatography 414: 335-342.
Swanson, S. P., Rood Jr., H. D., Behrens, J. C., and Sanders, P. E., 1987b. Preparation and characterization of the deepoxy trichothecenes: deepoxy HT-2, deepoxy T-2 triol, deepoxy T-2 tetraol, deepoxy 15-monoacetoxyscirpenol, and deepoxy scirpentriol. Applied and Environmental Microbiology 53: 2821-2826.
Swanson, S. p., Hagler, W. M., and Rood, H. D. (1984). Destruction of deoxynivalenol (vomitoxin) with sodium bisulphite. Abstracts of the Annual Meeting, American Society for Microbiology, Abstract O19.
Sweeney, M. J., & Dobson, A. D. W. (1998). Mycotoxin production by Aspergillus, Fusarium and Penicillium species. International Journal of Food Microbiology, 43, 141-158.
Tanaka, T., Yamamoto, S., Hasegawa, A., Aoki, N., Besling, J. R., Sugiura, Y., Ueno, Y., 1990. A survey of the natural occurrence of Fusarium mycotoxins, deoxynivalenol, nivalenol and zearalenone, in cereals harvested in The Netherlands. Mycopathologia 110, 19-22.
Todar, K. 2008. Nutrition and Growth of Bacteria in: Online Textbook of Bacteriology. Available at: http:// www.textbookofbacteriology.net (accesed on NOV/01/09)
Tomasevic-Canovic, M., Dakovic, A., Rottinghaus, G., Matijasevic, S., and Duricic, M. (2003). Surfactant modified zeolites—new efficient adsorbents for mycotoxins. Microporous and Mesoporous Materials 61, 173-180.
Trenholm, H. L., Charmley, L. L., Perlusky, D. B., and Warner, R. M. (1992). Washing procedures using water or sodium carbonate solutions for the decontamination of three cereals contaminated with deoxynivalenol and zearalenone. Journal of Agricultural and Food Chemistry 40, 2147-2151.
Trenholm, H. L., R. M. G. Hamilton, D. W. Friend, B. K. Thompson and K. E. Hartin. 1984. Feeding trials with vomitoxin (deoxynivalenol)- contaminated wheat: effects on swine, poultry, and dairy cattle. J. Amer. Vet. Med. Assoc. 185:527.
Ueno, Y., K. Nakayama, K. Ishii, F. Tashiro, Y. Minoda, T. Omori, and K. Komagata. 1983. Metabolism of T-2 toxin in Curtobacterium sp. strain 114-2. Appl. Environ. Microbiol. 46:120-127.
Ueno, Y. (1983). Trichothecene-producing fungi. In Developments in Food Science 4, Trichothecenes-Chemical, Biological and Toxicological Aspects (Y. Ueno, Ed.), pp. 73. Elsevier, Amsterdam, Netherlands.
Vesonder R F, Ciegler A, Jensen A H. 1973. Isolation of the emetic principle from Fusarium-infected corn. Applied Microbiology 26:1008-1010.
Vesonder R F, Hesseltine C W. 1981. Vomitoxin: natural occurrence on cereal grains and significance as a refusal and emetic factor to swine. Process Biochemistry 16:12, 14/15, 44.
Völkl, A., Vogler, B., Schollenberger, M. and Karlovsky, P., 2004. Microbial detoxification of mycotoxin deoxynivalenol. Journal of Basic Microbiology 44: 147-156.
Weinberg, D. 1978. Secondary metabolism: regulation by phosphate and trace elements. Folia Microbiol. 23 (6):496-504.
Westlake, K., Mackie, R. I. and Dutton, M. F., 1987. T-2 toxin metabolism by ruminal bacteria and its effect on their growth. Applied and Environmental Microbiology 53: 587-592.
Wu, F. 2007. Measuring the economic impacts of Fusarium toxins in animal feeds. J Animal Feed Science and Technology 137: 363-374.
Wu, F. (2004). Mycotoxin Risk Assessment for the Purpose of Setting International Regulatory Standards Environmental Science & Technology 38, 4049-4055.
Wu, F. (2006). A tale of two commodities: how EU mycotoxin regulations have hurt, or helped, food industries. The World Mycotoxin Forum®—the Fourth Conference Proceedings, pp. 30-32.
Young, J. C. (1986). Reduction in levels of deoxynivalenol in contaminated corn by chemical and physical treatment. Journal of Agricultural and Food Chemistry 34, 465-467.
Young, J. C., Blackwell, B. A., and ApSimon, J. W. (1986a). Alkaline Degradation of the Mycotoxin 4-Deoxynivalenol. Tetrahedron Letters 27, 1019-1022.
Young, J. C., Subryan, L. M., Potts, D., and McLaren, M. E. (1986b). Reduction in levels of deoxynivalenol in contaminated wheat by chemical and physical treatment. Journal of Agricultural and Food Chemistry 34, 461-465.
Young, J. C., Zhou, T., Yu, H., Zhu, H., and Gong, J. (2007). Degradation of trichothecene mycotoxins by chicken intestinal microbes. Food and Chemical Toxicology 45, 136-143.
Young, J. C., Zhu, H., and Zhou, T. (2006). Degradation of trichothecene mycotoxins by aqueous ozone. Food and Chemical Toxicology 44, 417-424.
Zabriskie, D. W., Armiger, W. B., Phillips, D. H., and Albano, P. A. 1980. Traders' guide to fermentation media formulation. Traders' Protein Co.: Memphis, Tenn.
Zhou T, He J and Gong J. 2008. Microbial transformation of trichothecene mycotoxins. World Mycotoxin Journal. Volume 1, Number 1. Pp 23-30.

TABLE 1
Comparison of soil suspension cultures from soils #11, 17, 21, 31, 110-1 and 165-2
with and without enrichment for activities of reduction of DON concentration

| | | | Without enrichment^{a} | | | | | |
|---|---|---|---|---|---|---|---|---|
| | | | | | Autoclaved soil | | Filtered cell-free soil | |
| | | | Soil suspension^{b} | | suspension^{b} | | suspension^{b} | |
| | | | Concentration | Reduction of | Concentration | Reduction of | Concentration | Reduction of |
| | | | of DON in | DON | of DON in | DON | of DON in | DON |
| Soil | | | culture | concentration | culture | concentration | culture | concentration |
| sample | Cultivation | Location | (μg/mL) | (%)^{c} | (μg/mL) | (%) | (μg/mL) | (%) |
| Soil | Wheat | Highway | 98.1 | 1.9 | 104.1 | −4.1 | 98.7 | 1.3 |
| #11 | | #12 at | | | | | | |
| | | Listowel | | | | | | |
| Soil | Alfalfa | Highway | 92.3 | 7.7 | 97.2 | 2.8 | 99.1 | 0.9 |
| #17 | | #12 | | | | | | |
| Soil | Corn | Highway | 97.0 | 3.0 | 97.6 | 2.4 | 104 | −4.0 |
| #21 | | #12/#8 | | | | | | |
| Soil | Grass | Highway | 93.9 | 6.1 | 99.5 | 0.5 | 102 | −2.0 |
| #31 | | #15 at | | | | | | |
| | | St. | | | | | | |
| | | Jacobs | | | | | | |
| Soil | Soil | — | 96.7 | 3.3 | 102.8 | −2.8 | 102 | −2.0 |
| #110-1 | mixture | | | | | | | |
| | of 110 | | | | | | | |
| | soils | | | | | | | |
| Soils | Soil | — | 93.6 | 6.4 | 103.3 | −3.3 | 96.9 | 3.1 |
| #165-2 | mixture | | | | | | | |
| | of 165 | | | | | | | |
| | soils | | | | | | | |
| PLSD_{(0.05)} | | | 9.4^{d} | | | | | |
| | | | With enrichment^{a} | | | | | |
| | | | | | Autoclaved soil | | Filtered cell-free soil | |
| | | | Soil suspension^{b} | | suspension^{b} | | suspension^{b} | |
| | | | Concentration | Reduction of | Concentration | Reduction of | Concentration | Reduction of |
| | | | of DON in | DON | of DON in | DON | of DON in | DON |
| Soil | | | culture | concentration | culture | concentration | culture | concentration |
| sample | Cultivation | Location | (μg/mL) | (%) | (μg/mL) | (%) | (μg/mL) | (%) |
| Soil | Wheat | Highway | 90.6 | 9.4 | 98.9 | 1.1 | 101 | −1.0 |
| #11 | | #12 at | | | | | | |
| | | Listowel | | | | | | |
| Soil | Alfalfa | Highway | 53.2 | 46.8 | 97.0 | 3.0 | 98.5 | 1.5 |
| #17 | | #12 | | | | | | |
| Soil | Corn | Highway | 86.4 | 13.6 | 98.1 | 1.9 | 99.4 | 0.6 |
| #21 | | #12/#8 | | | | | | |
| Soil | Grass | Highway | 43.2 | 56.8 | 97.9 | 2.1 | 102 | −2.0 |
| #31 | | #15 at | | | | | | |
| | | St. | | | | | | |
| | | Jacobs | | | | | | |
| Soil | Soil | — | 31.7 | 68.3 | 98.0 | 2.0 | 98.7 | 1.3 |
| #110-1 | mixture | | | | | | | |
| | of 110 | | | | | | | |
| | soils | | | | | | | |
| Soils | Soil | — | 24.5 | 75.5 | 98.3 | 1.7 | 97.6 | 2.4 |
| #165-2 | mixture | | | | | | | |
| | of 165 | | | | | | | |
| | soils | | | | | | | |

| | |
|---|---|
| PLSD_{(0.05)} | 9.4^{d} |

^{a}Original soil samples of soil #11, 17, 21, 31, 110-1 and 165-2 stored at 4° C. served as the soil samples without enrichment. Soil #11, 17, 21, 31, 110-1 and 165-2 incubated with F. graminearum + infested corn at 28° C. for 6 weeks served as soils with enrichment.
^{b}After cultured with soil suspension, autoclaved soil suspension and filtered cell-free soil suspension in MM medium, DON was added to each culture to make the final concentration as 100 μg/mL. The cultures were incubated at 28° C. for 3 d under aerobic condition.
^{c}Reduction of DON concentration was computed as (100 − Concentration of DON in culture)/Concentration of DON in culture × 100%.
^{d}Values of PLSD of the concentration of DON in culture (μg/mL) and Reduction of DON concentration (%) were the same.

TABLE 2
Comparison of activities of reduction of DON concentrations of
soil suspension cultures from different enrichment experiments
of soil #17 in CMB medium.

| | DON | |
|---|---|---|
| | concentration^{e} | Reduction of DON |
| Soil treatments | (μg/mL) | concentration (%) |
| Soil #17 without enrichment | 98.1 | 1.9 |
| Soil #17 enriched with | 5.2 | 94.8 |
| F. graminearum + infested corn^{a1} | | |
| Soil #17 enriched with | 99.0 | 1.0 |
| F. graminearum + infested corn, | | |
| then autoclaved^{a2} | | |
| Soil #17 enriched with | 99.9 | 0.1 |
| F. graminearum + infested corn, | | |
| then filtrated through 0.22 μm | | |
| MEC sterile syringe filter^{a3} | | |
| Soil #17 enriched with infested | 28.1 | 71.9 |
| corn (93 mg DON/g)^{b} | | |
| Soil #17 enriched with | 100.4 | −0.4 |
| F. graminearum^{c} | | |
| Autoclaved soil #17 enriched with | 106.8 | −6.8 |
| F. graminearum + infested corn^{d} | | |

| | |
|---|---|
| PLSD_{(0.05)} | 8.1^{f} |

^{a}Soil #17 enriched with F. graminearum + infested corn for 6 weeks. Three suspensions were obtained from this soil sample: a1 was the soil suspension from soil #17 after enrichment with F. graminearum + infested corn but without any further treatment; a2 was the autoclaved soil suspension from soil #17 after enrichment with F. graminearum + infested corn; a3 was the cell-free filtrate of the soil suspension from soil #17 after enrichment with F. graminearum + infested corn.
^{b}The soil suspension from soil #17 after enrichment with infested corn that contained 93 mg DON/g for 6 weeks.
^{c}The soil suspension from soil #17 after enrichment with F. graminearum for 6 weeks.
^{d}Soil #17 was autoclaved before enrichment with F. graminearum + infested corn for 6 weeks.
^{f}Values of PLSD_{(0.05)} of DON concentration (μg/mL) and reduction of DON concentration (%) were the same.

TABLE 3
Residual DON and its transformation products after cultured with
bacterial strain 040408-1 in a culture condition of: 100 μg/mL DON,
at 28° C., with shaking at 200 rpm for 72 h.

| | 3-epi-DON | Peak 5.0 | DON | 3-keto-DON |
|---|---|---|---|---|
| Medium | (μg/mL) | (μg/mL) | (μg/mL) | (μg/mL) |
| MMY | 24.7 | 0.0 | 30.5 | 0.0 |
| MM-Purdue | 0.0 | 0.0 | 93.9 | 0.0 |
| BYE | 58.0 | 0.0 | 14.6 | 3.82 |
| CMB | 49.2 | 4.9 | 4.58 | 4.61 |
| CMBPD | 22.2 | 0.0 | 81.1 | 0.0 |
| CMB/WO/Salt | 37.9 | 0.0 | 32.4 | 2.72 |
| Rice medium | 15.8 | 3.6 | 76.4 | 3.41 |
| Yeast + Glucose | 89.3 | 0.0 | 0.0 | 0.0 |
| Nutrient broth | 72.2 | 0.0 | 17.7 | 0.0 |
| TSB | 39.2 | 31.8 | 37.0 | 0.0 |
| Lauria Bertani | 34.5 | 0.0 | 31.0 | 0.0 |
| PLSD | 5.6 | 1.6 | 5.0 | 0.7 |

a. Peak number represented HPLC retention time (in minutes).
b. The UV maximum absorptions of 3-epi-DON, 5.0 and 3-keto-DON are in the range of 215~225 nm, which is close to maximum absorption of DON. Therefore, their concentrations can be calculated from peak areas to mass concentration using the standard curve of DON.

TABLE 4
IC₅₀ values for DON, 3-epi-DON and 3-keto-DON in 3T3

| | MTT bioassay | | | BrdU bioassay | | |
|---|---|---|---|---|---|---|
| | IC₅₀ | IC₅₀ relative to DON | | IC₅₀ | IC₅₀ relative to DON | |
| | μg/mL (95% Confidence | Mass | Molar | μg/mL (95% Confidence | Mass | Molar |
| Compound | Interval) | concentration¹ | concentration² | Interval) | concentration¹ | concentration² |
| DON | 0.409 (0.324, 0.518) | — | — | 0.238 (0.162, 0.349) | — | — |
| 3-epi-DON | 146 (100, 212) | 357 | 357 | 281 (156, 505) | 1181 | 1181 |
| 3-keto-DON | 1.24 (0.942, 1.62) | 3.03 | 3.05 | 1.08 (0.681, 1.72) | 4.54 | 4.57 |

TABLE 5
Body and organ weights of B6C3FI mice garaged with water, DON and 3-epi-DON
(All body weight data were expressed as mean (SEM) in g for n = 10 mice.)

| | | | | | | Relative | Relative | | |
|---|---|---|---|---|---|---|---|---|---|
| | | | | Feed | | spleen | thymus | Relative | Relative kidneys |
| | | | | efficacy | Relative | weight | weight | heart weight | weight |
| | | | Food | (SEM) | liver weight | (SEM) | (SEM) | (SEM) | (SEM) |
| | | | Consumption | (Body | (SEM) (liver | (spleen | (thymus | (heart | (kidneys |
| | Starting | Final body | during the | weight gain/ | weight/final | weight/final | weight/final | weight/final | weight/final |
| | body weight | weight | 14 days | Food | body | body | body | body | body |
| Treatment | (SEM) (g) | (SEM) (g) | (SEM) (g) | Consumption) | weight) | weight) | weight) | weight) | weight) |
| Control | 19.630 | 21.360 | 88.690 | 0.0385 | 0.0444 | 0.00347 | 0.00249 | 0.00437 | 0.0108 |
| | (0.340) | (0.477) | (2.236) | (0.00847) | (0.00138) | (0.000321) | (0.00014) | (0.00017) | (0.0003)^{a} |
| DON | 19.290 | 20.345 | 81.090 | 0.0252 | 0.0522 | 0.00280 | 0.00110 | 0.00474 | 0.0124 |
| 2 mg/kg bw | (0.265) | (0.297) | (2.523) | (0.00745) | (0.00166) | (0.000121) | (0.000083) | (0.00011) | (0.0003) |
| 3-epi-DON | 19.335 | 21.390 | 85.860 | 0.0475 | 0.0426 | 0.00295 | 0.00261 | 0.00447 | 0.0107 |
| 25 mg/kg bw | (0.194) | (0.363) | (1.660) | (0.00821) | (0.000914) | (0.000134) | (0.00014) | (0.000092) | (0.0002) |
| 3-epi-DON | 19.900 | 21.680 | 85.760 | 0.0413 | 0.0442 | 0.00297 | 0.00263 | 0.00439 | 0.0104 |
| 100 mg/kg bw | (0.242) | (0.384) | (1.661) | (0.00520) | (0.00119) | (0.000118) | (0.00010) | (0.000084) | (0.0002) |
| P value | 0.343 | 0.095 | 0.111 | 0.232 | <0.001 | 0.094 | <0.001 | 0.111 | <0.001 |

^{a}The unusual kidney of one of the control was not taken into account (n = 9).

TABLE 6
Media evaluated for the bacterium 040408-1 growth and
DON biotransformation¹.

| Nutrient | | Medium selected |
|---|---|---|
| Carbon source | Monosaccharide | Glucose (GLU) |
| | Disaccharide | Sucrose (SUC) |
| | Polysaccharide | Corn starch (STA) |
| Nitrogen source | Organic | Corn steep liquor (CSL) |
| | | Peptone (PEP) |
| | | Yeast extract (YEA) |
| | | Urea (URE) |
| | Inorganic | Ammonium sulphate (SUL) |
| | | Ammonium nitrate (NIT) |
| Control | Corn meal broth | |

¹All test media were evaluated with and without the addition of minerals.

TABLE 7
Growth and DON biotransformation of bacterial isolate
040408-1 at different cultivation temperatures¹

| Temperature | Growth | | DON biotransformation |
|---|---|---|---|
| (° C.) | (log CFU mL⁻¹)² | | (%)³ |
| 5 | 2.6 × | 10^{6 a} | 0.8 ^{a} |
| 10 | 1.2 × | 10^{7 a} | 0.8 ^{a} |
| 15 | 2.8 × | 10^{6 a} | 1.2 ^{a} |
| 20 | 1.0 × | 10^{9 b} | 82.0 ^{b} |
| 25 | 1.8 × | 10^{9 c} | 85.2 ^{b} |
| 30 | 2.5 × | 10^{9 d} | 88.0 ^{b} |
| 35 | 2.4 × | 10^{9 d} | 88.5 ^{b} |
| 40 | 1.6 × | 10^{9 bc} | 1.3 ^{a} |

¹Determined after 48 h in CMB at pH 6.9 and minerals added
²Values in the same column with different superscripts differ significantly according to Tukey's multiple range test (p-0.05)
³The percentage of DON biotransformation was estimated by subtracting the remaining DON after incubation from the initial concentration, multiplied × 100.

TABLE 8
Growth and DON biotransformation of bacterial isolate 040408-1
at different initial media pH¹

| Initial pH | Growth (log CFU mL⁻¹)² | DON Biotransformation (%)³ |
|---|---|---|
| 3.0 | 2.6 × 10^{6 a} | 2.0^{a} |
| 4.2 | 2.7 × 10^{6 a} | 1.6^{a} |
| 5.2 | 4.0 × 10^{7 a} | 2.3^{a} |
| 6.0 | 1.1 × 10^{9 b} | 77.5^{bc} |
| 7.1 | 1.0 × 10^{9 b} | 95.1^{c} |
| 8.4 | 1.1 × 10^{8 a} | 37.8^{b} |
| 9.3 | 8.8 × 10^{7 a} | 7.5^{a} |
| 10.2 | 9.5 × 10^{7 a} | 1.4^{a} |

¹Determined after 48 h shaken cultivation (200 rpm) at 28° C. in CMB plus minerals
²Values in the same column with different superscripts differ significantly according to Tukey's multiple range test (p-0.05)
³The percentage of DON biotransformation was estimated by subtracting the remaining DON after incubation from the initial concentration, multiplied × 100.

TABLE 9
Effect of carbon, nitrogen and minerals sources on growth and DON
biotransformation of bacterial isolate 040408-1¹

| | | | | | | DON | |
|---|---|---|---|---|---|---|---|
| | | | | Growth | | Biotransformation | |
| | | Initial pH² | | (log) CFU mL⁻¹)⁴ | | (%)⁵ | |
| | | without | Minerals | without | Minerals | without | Minerals |
| Nutrient (10 g L⁻¹) | | minerals | added³ | minerals | added | minerals | added |
| Carbon | Glucose | 5.0 | 7.5 | 1.6 × 10^{7a} | 1.2 × 10^{7a} | 0.8^{a} | 1.0^{a} |
| | Sucrose | 6.3 | 7.9 | 8.6 × 10^{6a} | 4.0 × 10^{7a} | 1.0^{a} | 8.3^{a} |
| | Corn starch | 5.2 | 7.7 | 2.8 × 10^{6a} | 9.9 × 10^{6a} | 0.7^{a} | 48.0^{ab} |
| Nitrogen | Corn steep liquor | 4.5 | 6.7 | 2.9 × 10^{9ab} | 3.5 × 10^{9b} | 17.1^{a} | 99.5^{b} |
| | Peptone | 7.2 | 7.6 | 1.5 × 10^{9ab} | 2.5 × 10^{9b} | 49.3^{ab} | 99.2^{b} |
| | Yeast extract | 6.0 | 7.2 | 5.3 × 10^{9b} | 8.7 × 10^{9c} | 87.7^{b} | 84.4^{b} |
| | Urea | 9.3 | 9.0 | 2.8 × 10^{6a} | 2.8 × 10^{6a} | 1.0^{a} | 1.0^{a} |
| | Ammonium sulphate | 4.4 | 6.9 | 2.9 × 10^{6a} | 3.0 × 10^{6a} | 1.4^{a} | 22.0^{a} |
| | Ammonium nitrate | 4.9 | 6.6 | 6.9 × 10^{6a} | 3.0 × 10^{6a} | 1.0^{a} | 18.6^{a} |
| Control | Corn meal Broth | | 6.9 | | 7.7 × 10^{9b} | | 97.8^{b} |
| | (control) | | | | | | |

¹Determined after 72 h in shaken culture (200 rpm) at 28° C.
²Values of media pH are shown.
³Minerals (per liter): K₂HPO₄, 1.0 g; MgSO₄, 0.5 g; K₂SO₄, 0.5 g; FeSO₄, 0.1 g and 0.07 MnSO₄, 0.07 g.
⁴Values in the same column with different superscripts differ significantly according to Tukey's multiple range test (p < 0.05).
⁵The percentage of DON biotransformation was estimated by subtracting the remaining DON after incubation from the initial concentration, multiplied × 100.

TABLE 10
Sequence similarity of 16S rRNA gene from bacterial strain 040408-1.

| | GenBank | Similarity |
|---|---|---|
| Description | Accession # | (%) |
| Devosia sp. 4_C16_46 16S ribosomal RNA | EF540511.1 | 97 |
| gene, partial sequence | | |
| Uncultured bacterium clone IYF22 16S | DQ984580.1 | 97 |
| ribosomal RNA gene, partial sequence | | |
| Uncultured alpha proteobacterium partial 16S | AJ532705.1 | 96 |
| rRNA gene, clone JG34-KF-258 | | |
| Antarctic bacterium A02 16S ribosomal RNA | EU636035.1 | 95 |
| gene, partial sequence | | |
| Devosia hwasunensis partial 16S rRNA | AM393883.1 | 95 |
| gene, type strain HST2-16T | | |
| Devosia insulae strain DS-56 16S ribosomal | EF012357.1 | 95 |
| RNA gene, partial sequence | | |
| Rhizobiales bacterium CSQ-10 16S ribosomal | EF512133.1 | 95 |
| RNA gene, partial sequence | | |
| Arctic sea ice bacterium ARK10037 16S | AF468359.1 | 94 |
| ribosomal RNA gene, partial sequence | | |
| Devosia subaequoris partial 16S rRNA gene, | AM293857.1 | 94 |
| strain type strain: HST3-14 | | |
| Devosia albogilva strain IPL15 16S ribosomal | EF433460.1 | 94 |
| RNA gene, partial sequence | | |
| Hyphomicrobiaceae bacterium H642 16S | GQ383921.1 | 94 |
| ribosomal RNA gene, partial sequence | | |

What is claimed is:

1. A bacterial isolate defined by accession number 040408-1 filed with the International Depository Authority of Canada.

2. A composition comprising bacteria as defined in claim 1.

3. The composition of claim 2, wherein the composition comprises a carrier.

4. The composition of claim 3, wherein the carrier is a food or food product contaminated or susceptible to contamination by trichothecene mycotoxins or organisms that produce trichothecene mycotoxins.

5. The composition of claim 4, wherein the trichothecene mycotoxin comprises deoxynivalenol (DON or vomitoxin).

6. A method of preventing or reducing mycotoxin contamination in a food or food product by treating the food or food product with bacteria as defined in claim 1.

7. The method as defined in claim 6, wherein the mycotoxin contamination comprises trichothecene mycotoxins.

8. The method of claim 7, wherein the trichothecene mycotoxins comprise deoxynivalenol (DON or vomitoxin).

9. A kit comprising bacteria as defined in claim 1 and one or more of the following:
a) one or more carriers for holding/retaining, suspending, diluting, adhering, enveloping, culturing, growing, or freezing/cryopreserving, the bacteria;
b) one or more devices for combining or formulating the bacteria with the one or more carriers of a)
c) one or more devices for treating a food or food product with the bacteria or a composition comprising the bacteria, and;
d) instructions for growing the bacteria, formulating the bacteria with the one or more carriers, using one or more devices for treating a food or food product, or a combination thereof.

10. A method of screening for microorganisms that are capable of reducing deoxynivalenol (DON or vomitoxin) comprising,
a) obtaining a soil sample;
b) culturing bacteria in the soil sample under conditions to enrich for bacteria that are capable of reducing deoxynivalenol (DON or vomitoxin);
c) isolating one or more single colonies of bacteria from the step of culturing (step b), and;
d) individually testing the one or more single colonies in an assay to confirm if the colony or colonies are capable of reducing deoxynivalenol (DON or vomitoxin).

11. The method of claim 10, further comprising culturing, purifying, isolating or any combination thereof the one or more single colonies that are capable of reducing deoxynivalenol (DON or vomitoxin).

12. The method of claim 10, wherein the step b) is preceded by a step of extracting bacteria from the soil sample.
